(19) United States
(12) Patent Application Publication
(10) Pub. No.: US 2005/0176801 A1
(43) Pub. Date: Aug. 11, 2005

(54) Acyl bicyclic derivatives of pyrrol

(76) Inventors: Martin John Slater, Hertfordshire (GB); Peter David Howes, Hertfordshire (GB)

Correspondence Address: DAVID J LEVY, CORPORATE INTELLECTUAL PROPERTY;GLAXOSMITHKLINE, FIVE MOORE DR., PO BOX 13398, RESEARCH TRIANGLE PARK, NC, 27709-3398, US

(21) Appl. No.: 10/514,898

(22) PCT Filed: May 15, 2003
(86) PCT No.: PCT/GB03/02105

(30) Foreign Application Priority Data
May 17, 2002 (GB) 0211418.9

(51) Int. Cl. A61K 31/407; C07D 487/02

(52) U.S. Cl. 514/412; 548/453

(57) ABSTRACT

The invention relates to anti-viral agents of Formula (I) 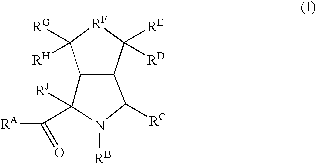 wherein:
R^{A} represents OR¹, NR¹R², or R¹ wherein R¹ and R² independently represent hydrogen, C₁₋₆alkyl, aryl, heteroaryl, arylalkyl or heteroarylalkyl; or R¹ and R² together with the nitrogen atom to which they are attached form a 5 or 6 membered saturated cyclic group;
R^{B} represents C(O)R³ wherein R³ represents aryl or heteroaryl;
R^{C} represents C₁₋₆alkyl, aryl, heteroaryl or heterocyclyl;
R^{D} represents hydrogen and R^{E} represents hydrogen, OR⁴ or SR⁴, or R^{D} and R^{E} together with the carbon atom to which they are attached form a carbonyl group or a thiocarbonyl group;
when R^{E} is hydrogen, OR⁴ or SR⁴, R^{G} and R^{H} are both hydrogen;
when R^{D} and R^{E} together with the carbon atom to which they are attached form a carbonyl group or a thiocarbonyl group, R^{G} represents hydrogen and R^{H} represents hydrogen, OR⁴ or SR⁴, or R^{G} and R^{H} together with the carbon atom to which they are attached form a carbonyl group or a thiocarbonyl group;
R⁴ represents hydrogen, C₁₋₆alkyl or aryl;
when R^{D} and R^{E} together with the carbon atom to which they are attached form a carbonyl group or a thiocarbonyl group, and R^{G} and R^{H} are both hydrogen or R^{G} and R^{H} together with the carbon atom to which they are attached form a carbonyl group or a thiocarbonyl group, then R^{F} represents O, S, NR⁵ or CR⁶R⁷, otherwise R^{F} represents CR⁶R⁷;
R⁵ represents hydrogen, C₁₋₆alkyl, arylalkyl or aryl;
R⁶ and R⁷ independently represent hydrogen, C₁₋₆alkyl, arylalkyl or heteroarylalkyl;
R^{J} represents hydrogen, C₁₋₆alkyl, heterocyclylalkyl, arylalkyl or heteroarylalkyl; and salts, solvates and enantiomers thereof;
provided that when R^{A} is OR¹ then R¹ is other than tert-butyl. A process for the preparation of compounds of Formula (I) and methods of using them in HCV treatment are provided.

### FIELD OF THE INVENTION

The present invention relates to novel acyl bicyclic derivatives of pyrrole useful as anti-viral agents. Specifically, the present invention involves novel HCV inhibitors.

### BACKGROUND OF THE INVENTION

In the US, an estimated 4.5 million Americans are chronically infected with HCV. Although only 30% of acute infections are symptomatic, greater than 85% of infected individuals develop chronic, persistent infection. Treatment costs for HCV infection have been estimated at $5.46 billion for the US in 1997. Worldwide over 200 million people are estimated to be infected chronically. HCV infection is responsible for 40-60% of all chronic liver disease and 30% of all liver transplants. Chronic HCV infection accounts for 30% of all cirrhosis, end-stage liver disease, and liver cancer in the U.S. The CDC estimates that the number of deaths due to HCV will minimally increase to 38,000/year by the year 2010.

Due to the high degree of variability in the viral surface antigens, existence of multiple viral genotypes, and demonstrated specificity of immunity, the development of a successful vaccine in the near future is unlikely. Alpha-interferon (alone or in combination with ribavirin) has been widely used since its approval for treatment of chronic HCV infection. However, adverse side effects are commonly associated with this treatment: flu-like symptoms, leukopenia, thrombocytopenia, depression from interferon, as well as anemia induced by ribavirin (Lindsay, K. L. (1997) Hepatology 26 (suppl 1): 71S-77S). This therapy remains less effective against infections caused by HCV genotype 1 (which constitutes ˜75% of all HCV infections in the developed markets) compared to infections caused by the other 5 major HCV genotypes. Unfortunately, only ˜50-80% of the patients respond to this treatment (measured by a reduction in serum HCV RNA levels and normalization of liver enzymes) and, of those treated, 50-70% relapse within 6 months of cessation of treatment. Recently, with the introduction of pegylated interferon, both initial and sustained response rates have improved substantially, and combination treatment of Peg-IFN with ribavirin constitutes the gold standard for therapy. However, the side effects asociated with combination therapy and the impaired response in patients with genotype 1 present opportunities for improvement in the management of this disease.

First identified by molecular cloning in 1989 (Choo, Q-L et al. (1989) Science 244:359-362), hepatitis C virus (HCV) is now widely accepted as the most common causative agent of post-transfusion non A, non-B hepatitis (NANBH) (Kuo, G et al. (1989) Science 244:362-364). Due to its genome structure and sequence homology, this virus was assigned as a new genus in the Flaviviridae family. Like the other members of the Flaviviridae, such as flaviviruses (e.g. yellow fever virus and Dengue virus types 1-4) and pestiviruses (e.g. bovine viral diarrhea virus, border disease virus, and classic swine fever virus) (Choo, Q-L et al. (1989) Science 244:359-3; Miller, R. H. and R. H. Purcell (1990) Proc. Natl. Acad. Sci. USA 87:2057-2061), HCV is an enveloped virus containing a single strand RNA molecule of positive polarity. The HCV genome is approximately 9.6 kilobases (kb) with a long, highly conserved, noncapped 5′ nontranslated region (NTR) of approximately 340 bases which functions as an internal ribosome entry site (IRES) (Wang C Y et al. ‘An RNA pseudoknot is an essential structural element of the internal ribosome entry site located within the hepatitis C virus 5′ noncoding region’ [Article] Rna-A Publication of the Rna Society. 1(5):526-537, 1995 July). This element is followed by a region which encodes a single long open reading frame (ORF) encoding a polypeptide of −3000 amino acids comprising both the structural and nonstructural viral proteins.

Upon entry into the cytoplasm of the cell, this RNA is directly translated into a polypeptide of ˜3000 amino acids comprising both the structural and nonstructural viral proteins. This large polypeptide is subsequently processed into the individual structural and nonstructural proteins by a combination of host and virally-encoded proteinases (Rice, C. M. (1996) in B. N. Fields, D. M. Knipe and P. M. Howley (eds) Virology 2^{nd} Edition, p 931-960; Raven Press, N.Y.). Following the termination codon at the end of the long ORF, there is a 3′ NTR which roughly consists of three regions: an ˜40 base region which is poorly conserved among various genotypes, a variable length poly(U)/polypyrimidine tract, and a highly conserved 98 base element also called the “3′ X-tail” (Kolykhalov, A. et al (1996) J. Virology 70:3363-3371; Tanaka, T. et al. (1995) Biochem Biophys. Res. Commun. 215:744-749; Tanaka, T. et al. (1996) J. Virology 70:3307-3312; Yamada, N. et al. (1996) Virology 223:255-261). The 3′ NTR is predicted to form a stable secondary structure which is essential for HCV growth in chimps and is believed to function in the initiation and regulation of viral RNA replication.

The NS5B protein (591 amino acids, 65 kDa) of HCV (Behrens, S. E. et al. (1996) EMBO J. 15:12-22), encodes an RNA-dependent RNA polymerase (RdRp) activity and contains canonical motifs present in other RNA viral polymerases. The NS5B protein is fairly well conserved both intra-typically (˜95-98% amino acid (aa) identity across 1b isolates) and inter-typically (˜85% aa identity between genotype 1a and 1b isolates). The essentiality of the HCV NS5B RdRp activity for the generation of infectious progeny virions has been formally proven in chimpanzees (A. A. Kolykhalov et al. (2000) Journal of Virology, 74(4):2046-2051). Thus, inhibition of NS5B RdRp activity (inhibition of RNA replication) is predicted to cure HCV infection.

Based on the foregoing, there exists a significant need to identify synthetic or biological compounds for their ability to inhibit HCV.

Certain bicyclic compounds have been synthesised and disclosed in the prior art; some of these compounds have been shown to have utility in a number of therapeutic areas, as outlined below.

B. Henkel et al. (2000) Bioorganic & Medicinal Chemistry Letters 10:975-977 relates to a stereo-random synthesis of highly functionalised proline analogues by azomethine cycloaddition. It specifically discloses several 1,5-diazobicyclo[3.3.0]octane-2-carboxylic acid analogues of proline, but no therapeutic use is mentioned.

A. J. Bicknell et al. (1996) Bioorganic & Medicinal Chemistry Letters 6(20):2441-2444 similarly relates to the synthesis of a highly functionalised rigid template by solid phase azomethine ylide cycloaddition. It specifically discloses several 1,5-diazobicyclo[3.3.0]octane-2-carboxylic adds, but no therapeutic use is mentioned.

WO 00/18772 A1 relates to condensed imidazolidinones as tRNA synthetase inhibitors. It generically discloses a group of bicyclic compounds which exhibit tRNA synthetase inhibition.

U.S. Pat. No. 5,231,083 (equivalent to DE 3926606) discloses compounds possessing certain bicylic systems having utility for the treatment of cardiac and of vascular hypertrophy and/or hyperplasia.

WO 02/18369 A2 discloses peptidometric compounds possessing certain bicyclic systems having utility as protease inhibitors, including as hepatitis C virus NS3 protease inhibitors.

WO 02/055491 A2 discloses 1,2-disubstituted cyclic matrix metalloproteases and tumour necrosis factor (TNF)-α inhibitors, which have utility in pathological conditions such as osteo- and rheumatiod arthritis, certain ulcerations, tumour metastasis or invasion, periodontal disease and bone disease.

The present invention discloses certain bicyclic compounds having potential to inhibit NS5B HCV polymerase activity and therefore having utility in the treatment of viral infection, especially HCV infection.

### SUMMARY OF THE INVENTION

The present invention involves compounds represented hereinbelow, pharmaceutical compositions comprising such compounds and use of the present compounds in treating viral infection, especially HCV infection.

### DETAILED DESCRIPTION OF THE INVENTION

The present invention provides compounds of Formula (I): 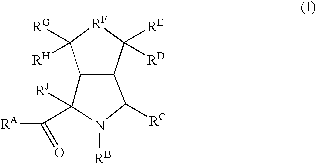
wherein:
R^{A} represents OR¹, NR¹R², or R¹ wherein R¹ and R² independently represent hydrogen, C₁₋₆alkyl, aryl, heteroaryl, arylalkyl or heteroarylalkyl; or R¹ and R² together with the nitrogen atom to which they are attached form a 5 or 6 membered saturated cyclic group;
R^{B} represents C(O)R³ wherein R³ represents aryl or heteroaryl;
R^{C} represents C₁₋₆alkyl, aryl, heteroaryl or heterocyclyl;
R^{D} represents hydrogen and R^{E} represents hydrogen, OR⁴ or SR⁴, or R^{D} and R^{E} together with the carbon atom to which they are attached form a carbonyl group or a thiocarbonyl group;
when R^{E} is hydrogen, OR⁴ or SR⁴, R^{G} and R^{H} are both hydrogen;
when R^{D} and R^{E} together with the carbon atom to which they are attached form a carbonyl group or a thiocarbonyl group, R^{G} represents hydrogen and R^{H} represents hydrogen, OR⁴ or SR⁴, or R^{G} and R^{H} together with the carbon atom to which they are attached form a carbonyl group or a thiocarbonyl group;
R⁴ represents hydrogen, C₁₋₆alkyl or aryl;
when R^{D} and R^{E} together with the carbon atom to which they are attached form a carbonyl group or a thiocarbonyl group, and R^{G} and R^{H} are both hydrogen or R^{G} and R^{H} together with the carbon atom to which they are attached form a carbonyl group or a thiocarbonyl group, then R^{F} represents O, S, NR⁵ or CR⁶R⁷, otherwise R^{F} represents CR⁶R⁷;
R⁵ represents hydrogen, C₁₋₆alkyl, arylalkyl or aryl;
R⁶ and R⁷ independently represent hydrogen, C₁₋₆alkyl, arylalkyl or heteroarylalkyl;
R^{J} represents hydrogen, C₁₋₆alkyl, heterocyclylalkyl, arylalkyl or heteroarylalkyl;
and salts, solvates and enantiomers thereof;
provided that when R^{A} is OR¹ then R¹ is other than tert-butyl, for use in medical therapy.

Thus, there is provided as one aspect of the present invention a compound of formula (I) or a physiologically acceptable salt, solvate or enantiomer thereof for use in human or veterinary medical therapy, particularly in the treatment of viral infection, particularly HCV infection.

It will be appreciated that reference herein to treatment includes, but is not limited to prevention, retardation, prophylaxis, therapy and cure of the disease. It will further be appreciated that references herein to treatment or prophylaxis of HCV infection includes treatment or prophylaxis of HCV-associated disease such as liver fibrosis, cirrhosis and hepatocellular carcinoma.

According to another aspect of the invention, there is provided the use of a compound of formula (I) or a physiologically acceptable salt, solvate or enantiomer thereof in the manufacture of a medicament for the treatment of viral infection, particularly HCV infection.

In a further aspect of the invention, there is provided a method for the treatment of a human or animal subject with viral infection, particularly HCV infection, which method comprises administering to said human or animal subject an effective amount of a compound of formula (I) or a physiologically acceptable salt, solvate or enantiomer thereof.

In another aspect of the invention, there is provided novel compounds of Formula (I), represented by Formula (A) 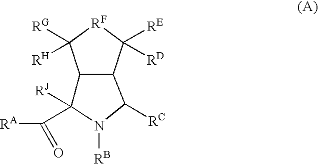
wherein:
R^{A} represents OR¹, NR¹R², or R¹ wherein R¹ and R² independently represent hydrogen, C₁₋₆alkyl, aryl, heteroaryl, arylalkyl or heteroarylalkyl; or R¹ and R² together with the nitrogen atom to which they are attached form a 5 or 6 membered saturated cyclic group;
R^{B} represents C(O)R³ wherein R³ represents aryl or heteroaryl;
R^{C} represents C₁₋₆alkyl, aryl, heteroaryl or heterocyclyl;
R^{D} represents hydrogen and R^{E} represents hydrogen, OR⁴ or SR⁴, or R^{D} and R^{E} together with the carbon atom to which they are attached form a carbonyl group or a thiocarbonyl group;
when R^{E} is hydrogen, OR⁴ or SR⁴, R^{G} and R^{H} are both hydrogen;
when R^{D} and R^{E} together with the carbon atom to which they are attached form a carbonyl group or a thiocarbonyl group, R^{G} represents hydrogen and R^{H} represents hydrogen, OR⁴ or SR⁴, or R^{G} and R^{H} together with the carbon atom to which they are attached form a carbonyl group or a thiocarbonyl group;
R⁴ represents hydrogen, C₁₋₆alkyl or aryl;
when R^{D} and R^{E} together with the carbon atom to which they are attached form a carbonyl group or a thiocarbonyl group, and R^{G} and R^{H} are both hydrogen or R^{G} and R^{H} together with the carbon atom to which they are attached form a carbonyl group or a thiocarbonyl group, then R^{F} represents O, S, NR⁵ or CR⁶R⁷, otherwise R^{F} represents CR⁶R⁷;
R⁵ represents hydrogen, C₁₋₆alkyl, arylalkyl or aryl;
R⁶ and R⁷ independently represent hydrogen, C₁₋₆alkyl, arylalkyl or heteroarylalkyl;
R^{J} represents hydrogen, C₁₋₆alkyl, heterocyclylalkyl, arylalkyl or heteroarylalkyl;
and salts, solvates and enantiomers thereof; provided that
i) when R^{A} is OR¹ then R¹ is other than tert-butyl, and
ii) a compound of Formula (A) is other than
rel-(3aS,6aR)-1-((1H-imidazol-5-yl)methyl)-2-(cyclopropylbenzoyl)-3-(4-methoxyphenyl)-5-methyl-octahydro-4,6-dioxo-pyrrolo[3,4-c]pyrrole-1-carboxylic acid; or
rel-(1S,3R,3aS,6aR)-1-methyl-2-benzoyl-3-(4-methoxyphenyl)-5-phenyl-octahydro-4,6-dioxo-pyrrolo[3,4-c]pyrrole-1-carboxylic acid; or
rel-(1S,3R,3aS,6aR)-1-methyl-2-benzoyl-3-(4-nitrophenyl)-5-methyl-octahydro-4,6-dioxo-pyrrolo[3,4-c]pyrrole-1-carboxylic acid; or
rel-(1S,3R,3aS,6aR)-1-benzyl-2-(phenylethanoyl)-3-(4-methoxyphenyl)-5-methyl-octahydro-4,6-dioxo-pyrrolo[3,4-c]pyrrole-1-carboxylic acid; or
rel-(1S,3R,3aS,6aR)-1-(2-methylpropyl)-2-ethanoyl-3-(4-methoxyphenyl)-5-methyl-octahydro-4,6-dioxo-pyrrolo[3,4-c]pyrrole-1-carboxylic acid; or
rel-(1S,3R,3aS,6aR)-1-methyl-2-ethanoyl-3-(4-methoxyphenyl)-5-methyl-octahydro-4,6-dioxo-pyrrolo[3,4-c]pyrrole-1-carboxylic acid.

In one group of compounds of Formula (I), R^{A} represents OR¹ or R¹ wherein R¹ is hydrogen, or R^{A} represents NR¹R² wherein R¹ and R² are as defined in Formula (I) above, and R^{B}, R^{C}, R^{D}, R^{E}, R^{F}, R^{G} and R^{H} are as defined in Formula (I) above.

In another group of compounds of Formula (I), when R^{D} represents hydrogen; R^{E} represents hydrogen, OR⁴ or SR⁴; R^{F} represents CR⁶R⁷; R^{G} represents hydrogen; R^{H} represents hydrogen and R^{A}, R^{B} and R^{J} are as defined in Formula (I) above, then R^{C} represents aryl, heteroaryl or heterocyclyl.

In one group of compounds of Formula (A), R^{A} represents OR¹ or R¹ wherein R¹ is hydrogen, or R^{A} represents NR¹R² wherein R¹ and R² are as defined in Formula (A) above, and R^{B}, R^{C}, R^{D}, R^{E}, R^{F}, R^{G} and R^{H} are as defined in Formula (A) above.

In another group of compounds of Formula (A), when R^{D} represents hydrogen; R^{E} represents hydrogen, OR⁴ or SR⁴; R^{F} represents CR⁶R⁷; R^{G} represents hydrogen; R^{H} represents hydrogen and R^{A}, R^{B} and R^{J} are as defined in Formula (A) above, then R^{C} represents aryl, heteroaryl or heterocyclyl.

Preferably, the ring junction in Formula (I) is cis-fused, as shown in Formula (Ia):

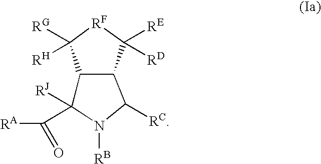

It will be appreciated by the person skilled in the art that Formula (Ia) indicates the relative stereochemistry of the two chiral centres at the ring junction.

In another aspect of the invention, there is provided compounds of Formula (I) represented by Formula (Ib): 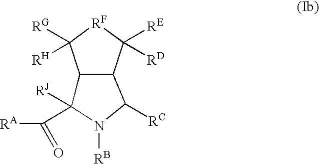
wherein:
R^{A} represents OR¹, NR¹R², or R¹ wherein R¹ and R² independently represent C₁₋₆alkyl, aryl, heteroaryl, arylalkyl or heteroarylalkyl; or R¹ and R² together with the nitrogen atom to which they are attached form a 5 or 6 membered saturated cyclic group;
R^{B} represents C(O)R³ wherein R³ represents C₁₋₆alkyl, aryl, heteroaryl, arylalkyl or heteroarylalkyl;
R^{C} represents C₁₋₆alkyl, aryl, heteroaryl or heterocyclyl;
R^{D} represents hydrogen and R^{E} represents hydrogen, OR⁴ or SR⁴, or R^{D} and R^{E} together with the carbon atom to which they are attached form a carbonyl group or a thiocarbonyl group;
when R^{E} is hydrogen, OR⁴ or SR⁴, R^{G} and R^{H} are both hydrogen;
when R^{D} and R^{E} together with the carbon atom to which they are attached form a carbonyl group or a thiocarbonyl group, R^{G} represents hydrogen and R^{H} represents hydrogen, OR⁴ or SR⁴, or R^{G} and R^{H} together with the carbon atom to which they are attached form a carbonyl group or a thiocarbonyl group;
R⁴ represents hydrogen, C₁₋₆alkyl or aryl;
when R^{D} and R^{E} together with the carbon atom to which they are attached form a carbonyl group or a thiocarbonyl group, and R^{G} and R^{H} are both hydrogen or R^{G} and R^{H} together with the carbon atom to which they are attached form a carbonyl group or a thiocarbonyl group, then R^{F} represents O, S, NR⁵ or CR⁶R⁷, otherwise R^{F} represents CR⁶R⁷;
R⁵ represents hydrogen, C₁₋₆alkyl, arylalkyl or aryl;
R⁶ and R⁷ independently represent hydrogen, C₁₋₆alkyl, arylalkyl or heteroarylalkyl;
R^{J} represents hydrogen, C₁₋₆alkyl, heterocyclylalkyl, arylalkyl or heteroarylalkyl;
and salts and solvates thereof;
provided that when R^{A} is OR¹ then R¹ is other than tert-butyl.

In yet another aspect of the invention, there is provided compounds of Formula (I) represented by Formula (Ic): 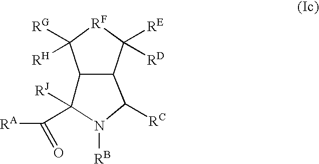
wherein:
R^{A} represents OH;
R^{B} represents C(O)R³ wherein R³ represents C₁₋₆alkyl, aryl, heteroaryl, arylalkyl or heteroarylalkyl;
R^{C} represents C₁₋₆alkyl, aryl, heteroaryl or heterocyclyl;
R^{D} represents hydrogen and R^{E} represents hydrogen, OR⁴ or SR⁴, or R^{D} and R^{E} together with the carbon atom to which they are attached form a carbonyl group or a thiocarbonyl group;
when R^{E} is hydrogen, OR⁴ or SR⁴, R^{G} and R^{H} are both hydrogen;
when R^{D} and R^{E} together with the carbon atom to which they are attached form a carbonyl group or a thiocarbonyl group, R^{G} represents hydrogen and R^{H} represents hydrogen, OR⁴ or SR⁴, or R^{G} and R^{H} together with the carbon atom to which they are attached form a carbonyl group or a thiocarbonyl group;
R⁴ represents hydrogen, C₁₋₆alkyl or aryl;
when R^{D} and R^{E} together with the carbon atom to which they are attached form a carbonyl group or a thiocarbonyl group, and R^{G} and R^{H} are both hydrogen or R^{G} and R^{H} together with the carbon atom to which they are attached form a carbonyl group or a thiocarbonyl group, then R^{F} represents O, S, NR⁵ or CR⁶R⁷, otherwise R^{F} represents CR⁶R⁷;
R⁵ represents hydrogen, C₁₋₆alkyl, arylalkyl or aryl;
R⁶ and R⁷ independently represent hydrogen, C₁₋₆alkyl, arylalkyl or heteroarylalkyl;
R^{J} represents hydrogen, C₁₋₆alkyl, heterocyclylalkyl, arylalkyl or heteroarylalkyl;
and salts and solvates thereof.

In yet another aspect of the invention, there is provided compounds of Formula (I) represented by Formula (Id): 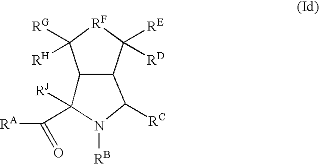
wherein:
R^{A} represents OR¹, NR¹R², or R¹ wherein R¹ and R² independently represent C₁₋₆alkyl, aryl, heteroaryl, arylalkyl or heteroarylalkyl; or R¹ and R² together with the nitrogen atom to which they are attached form a 5 or 6 membered saturated cyclic group;
R^{B} represents C(O)R³ wherein R³ represents aryl or heteroaryl;
R^{C} represents C₁₋₆alkyl, aryl, heteroaryl or heterocyclyl;
R^{D} represents hydrogen and R^{E} represents hydrogen, OR⁴ or SR⁴, or R^{D} and R^{E} together with the carbon atom to which they are attached form a carbonyl group or a thiocarbonyl group;
when R^{E} is hydrogen, OR⁴ or SR⁴, R^{G} and R^{H} are both hydrogen;
when R^{D} and R^{E} together with the carbon atom to which they are attached form a carbonyl group or a thiocarbonyl group, R^{G} represents hydrogen and R^{H} represents hydrogen, OR⁴ or SR⁴, or R^{G} and R^{H} together with the carbon atom to which they are attached form a carbonyl group or a thiocarbonyl group;
R⁴ represents hydrogen, C₁₋₆alkyl or aryl;
when R^{D} and R^{E} together with the carbon atom to which they are attached form a carbonyl group or a thiocarbonyl group, and R^{G} and R^{H} are both hydrogen or R^{G} and R^{H} together with the carbon atom to which they are attached form a carbonyl group or a thiocarbonyl group, then R^{F} represents O, S, NR⁵ or CR⁶R⁷, otherwise R^{F} represents CR⁶R⁷;
R⁵ represents hydrogen, C₁₋₆alkyl, arylalkyl or aryl;
R⁶ and R⁷ independently represent hydrogen, C₁₋₆alkyl, arylalkyl or heteroarylalkyl;
R^{J} represents hydrogen, C₁₋₆alkyl, heterocyclyalkyl, arylalkyl or heteroarylalkyl;
and salts and solvates thereof;
provided that when R^{A} is OR¹ then R¹ is other than tert-butyl, and
i) when R^{A} is OR¹ then R¹ is other than tert-butyl, and
ii) a compound of Formula (Id) is other than
rel-(3aS,6aR)-1-((1H-imidazol-5-yl)methyl)-2-(cyclopropylbenzoyl)-3-(4-methoxyphenyl)-5-methyl-octahydro-4,6-dioxo-pyrrolo[3,4-c]pyrrole-1-carboxylic acid; or
rel-(1S,3R,3aS,6aR)-1-methyl-2-benzoyl-3-(4-methoxyphenyl)-5-phenyl-octahydro-4,6-dioxo-pyrrolo[3,4-c]pyrrole-1-carboxylic acid; or
rel-(1S,3R,3aS,6aR)-1-methyl-2-benzoyl-3-(4-nitrophenyl)-5-methyl-octahydro-4,6-dioxo-pyrrolo[3,4-c]pyrrole-1-carboxylic acid; or
rel-(1S,3R,3aS,6aR)-1-benzyl-2-(phenylethanoyl)-3-(4-methoxyphenyl)-5-methyl-octahydro-4,6-dioxo-pyrrolo[3,4-c]pyrrole-1-carboxylic acid; or
rel-(1S,3R,3aS,6aR)-1-(2-methylpropyl)-2-ethanoyl-3-(4-methoxyphenyl)-5-methyl-octahydro-4,6-dioxo-pyrrolo[3,4-c]pyrrole-1-carboxylic acid; or
rel-(1S,3R,3aS,6aR)-1-methyl-2-ethanoyl-3-(4-methoxyphenyl)-5-methyl-octahydro-4,6-dioxo-pyrrolo[3,4-c]pyrrole-1-carboxylic acid.

In yet another aspect of the invention, there is provided compounds of Formula (I) represented by Formula (Ie): 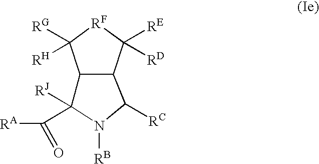
wherein:
R^{A} represents OH;
R^{B} represents C(O)R³ wherein R³ represents aryl or heteroaryl;
R^{C} represents C₁₋₆alkyl, aryl, heteroaryl or heterocyclyl;
R^{D} represents hydrogen and R^{E} represents hydrogen, OR⁴ or SR⁴, or R^{D} and R^{E} together with the carbon atom to which they are attached form a carbonyl group or a thiocarbonyl group;
when R^{E} is hydrogen, OR⁴ or SR⁴, R^{G} and R^{H} are both hydrogen;
when R^{D} and R^{E} together with the carbon atom to which they are attached form a carbonyl group or a thiocarbonyl group, R^{G} represents hydrogen and R^{H} represents hydrogen, OR⁴ or SR⁴, or R^{G} and R^{H} together with the carbon atom to which they are attached form a carbonyl group or a thiocarbonyl group;
R⁴ represents hydrogen, C₁₋₆alkyl or aryl;
when R^{D} and R^{E} together with the carbon atom to which they are attached form a carbonyl group or a thiocarbonyl group, and R^{G} and R^{H} are both hydrogen or R^{G} and R^{H} together with the carbon atom to which they are attached form a carbonyl group or a thiocarbonyl group, then R^{F} represents O, S, NR⁵ or CR⁶R⁷, otherwise R^{F} represents CR⁶R⁷;
R⁵ represents hydrogen, C₁₋₆alkyl, arylalkyl or aryl;
R⁶ and R⁷ independently represent hydrogen, C₁₋₆alkyl, arylalkyl or heteroarylalkyl;
R^{J} represents hydrogen, C₁₋₆alkyl, heterocyclylalkyl, arylalkyl or heteroarylalkyl;
and salts and solvates thereof;
provided that when R^{A} is OR¹ then R¹ is other than tert-butyl, and
i) when R^{A} is OR¹ then R¹ is other than tert-butyl, and
ii) a compound of Formula (Ie) is other than
rel-(3aS,6aR)-1-((1H-imidazol-5-yl)methyl)-2-(cyclopropylbenzoyl)-3-(4-methoxyphenyl)-5-methyl-octahydro-4,6-dioxo-pyrrolo[3,4-c]pyrrole-1-carboxylic acid; or
rel-(1S,3R,3aS,6aR)-1-methyl-2-benzoyl-3-(4-methoxyphenyl)-5-phenyl-octahydro-4,6-dioxo-pyrrolo[3,4-c]pyrrole-1-carboxylic acid; or
rel-(1S,3R,3aS,6aR)-1-methyl-2-benzoyl-3-(4-nitrophenyl)-5-methyl-octahydro-4,6-dioxo-pyrrolo[3,4-c]pyrrole-1-carboxylic acid; or
rel-(1S,3R,3aS,6aR)-1-benzyl-2-(phenylethanoyl)-3-(4-methoxyphenyl)-5-methyl-octahydro-4,6-dioxo-pyrrolo[3,4-c]pyrrole-1-carboxylic acid; or
rel-(1S,3R,3aS,6aR)-1-(2-methylpropyl)-2-ethanoyl-3-(4-methoxyphenyl)-5-methyl-octahydro-4,6-dioxo-pyrrolo[3,4-c]pyrrole-1-carboxylic acid; or
rel-(1S,3R,3aS,6aR)-1-methyl-2-ethanoyl-3-(4-methoxyphenyl)-5-methyl-octahydro-4,6-dioxo-pyrrolo[3,4-c]pyrrole-1-carboxylic acid.

The following R groups are preferred, where applicable, in respect of each of Formulae I, Ia, Ib, Ic, Id, Ie and A:
Preferably, R^{A} is OR¹, more preferably R^{A} is OH;
Preferably, R³ is aryl, more preferably R³ is phenyl, optionally substituted by halo, C₁₋₆alkyl or C₁₋₃alkoxy. Especially preferred is 4-tert-butylphenyl, optionally 3-substituted by halo, C₁₋₃alkyl or C₁₋₃alkoxy, especially bromo, chloro, methyl or methoxy; most preferably R³ is 4-tert-butylphenyl, 3-bromo-4-tert-butylphenyl or 4-tert-butyl-3-methoxyphenyl;
Preferably, R^{C} represents C₁₋₆alkyl, aryl or heteroaryl; more preferably R^{C} represents heteroaryl, especially preferred are pyridin-2-yl, pyrazin-2-yl, 1,3-thiazol-2-yl, 5-methyl-1,3-thiazol-2-yl or 1,3-thiazol-4-yl; most preferred are R^{C} is 1,3-thiazol-2-yl, 5-methyl-1,3-thiazol-2-yl or pyridin-2-yl;
Preferably, R^{D} and R^{E} together with the carbon atom to which they are attached form a carbonyl group;
Preferably, R^{G} and R^{H} are both hydrogen or R^{G} and R^{H} together with the carbon atom to which they are attached form a carbonyl group;
Preferably, R^{F} is O, NR⁵ or CR⁶R⁷, more preferably R^{F} is O, NCH₃, N-benzyl, CH₂ or C(CH₃)₂;
Preferably, R^{J} is C₁₋₆alkyl, more preferably R^{J} is isobutyl.

It is to be understood that the present invention covers all combinations of suitable, convenient and preferred groups described herein.

As used herein, “alkyl” refers to an optionally substituted hydrocarbon group. The alkyl hydrocarbon group may be linear, branched or cyclic, saturated or unsaturated. Where the alkyl hydrocarbon group is cyclic, it will be understood that there will be a minimum of 3 carbon atoms in the group. Preferably, the group is saturated. Preferred alkyl moieties are C₁₋₄alkyl. Optional subsituents include C₁₋₆alkyl, halo, OR⁸, C(O)NR⁹R¹⁰, CO₂R³, NR⁹R¹⁰, NHC(O)R³, NHCO₂R³, NHC(O)NR¹R², SO₂NR¹R², SO₂R³, nitro, oxo, and heterocyclyl.

R⁸ represents hydrogen, C₁₋₆alkyl, arylalkyl, or heteroarylalkyl; R⁹ and R¹⁰ are independently selected from hydrogen, C₁₋₆alkyl, aryl and heteroaryl.

As used herein, “aryl” refers to an optionally substituted aromatic group with at least one ring having a conjugated pi-electron system, containing up to two conjugated or fused ring systems. “Aryl” includes carbocyclic aryl and biaryl groups, all of which may be optionally substituted. Preferred “aryl” moieties are C₆₋₁₄aryl. Preferred “aryl” moieties are unsubstituted, monosubstituted, disubstituted or trisubstituted phenyl. Preferred “aryl” substituents are selected from the group consisting of C₁₋₆alkyl, halo, OR⁸, C(O)NR⁹R¹⁰, CO₂R³, NR⁹R¹⁰, NHC(O)R³, NHCO₂R³, NHC(O)NR¹R², SO₂NR¹R², SO₂R³, nitro, heterocyclyl, OC₁₋₄alkyl, CF₃, pyridine and phenyl.

As used herein, “heteroaryl” refers to an optionally substituted, 5 or 6 membered, aromatic group comprising one to four heteroatoms selected from N, O and S, with at least one ring having a conjugated pi-electron system, containing up to two conjugated or fused ring systems. Preferred “heteroaryl” moieties are unsubstituted, monosubstituted, disubstituted or trisubstituted thienyl and thiazolyl. Preferred “heteroaryl” substituents are selected from the group consisting of C₁₋₆alkyl, halo, OR⁸, C(O)NR⁹R¹⁰, CO₂R³, NR⁹R¹⁰, NHC(O)R³, NHCO₂R³, NHC(O)NR¹R², SO₂NR¹R², SO₂R³, nitro, heterocyclyl, OC₁₋₄alkyl, CF₃, pyridine and phenyl.

As used herein, “heterocyclic”, “heterocyclyl” and “5 or 6 membered saturated cyclic group” refer to an optionally substituted, 5 or 6 membered, saturated cyclic hydrocarbon group containing one to four heteroatoms selected from N, optionally substituted by hydrogen, C₁₋₆alkyl, C(O)R³, SO₂R³, aryl or heteroaryl; O; and S, optionally substituted by one or two oxygen atoms.

Preferred compounds useful in the present invention are selected from the group consisting of:
rel-(1S,3R,3aS,6aR)-1-Isobutyl-2-(4-tert-butylbenzoyl)-3-(1,3-thiazol-2-yl)-5-propyl-octahydro-4,6-dioxo-pyrrolo[3,4-c]pyrrole-1-carboxylic acid;
rel-(1S,3R,3aS,6aR)-1-Isobutyl-2-(4-tert-butylbenzoyl)-3-(1,3-thiazol-2-yl)-5-benzyl-octahydro-4,6-dioxo-pyrrolo[3,4-c]pyrrole-1-carboxylic acid;
rel-(1S,3R,3aS,6aR)-1-Isobutyl-2-(4-tert-butylbenzoyl)-3-(1,3-thiazol-2-yl)-5-methyl-octahydro-4,6-dioxo-pyrrolo[3,4-c]pyrrole-1-carboxylic acid;
rel-(1S,3R,3aS,6aR)-1-Isobutyl-2-(4-tert-butylbenzoyl)-3-(1,3-thiazol-2-yl)-octahydro-4-oxo-cyclopenta[c]pyrrole-1-carboxylic acid;
rel-(3aR,4S,6R,6aS)-4-Isobutyl-5-(4-tert-butylbenzoyl)-6-(1,3-thiazol-2-yl)-hexahydro-furo[3,4-c]pyrrol-1-oxo-4-carboxylic acid;
rel-(1S,3R,3aS,6aR)-1-Isobutyl-2-(3-methoxy-4-ert-butylbenzoyl)-3-(1,3-thiazol-2-yl) octahydro-4-oxo-cyclopenta[c]pyrrole-1-carboxylic acid;
rel-(1S,3R,3aS,6aR)-1 Isobutyl-2-(3-bromo-4-tert-butylbenzoyl)-3-(1,3-thiazol-2-yl)-5-propyl-octahydro-4,6-dioxo-pyrrolo[3,4-c]pyrrole-1-carboxylic acid;
rel-(1S,3R,3aS,6aR)-1 Isobutyl-2-(3-bromo-4-tert-butylbenzoyl)-3-(1,3-pyridin-2-yl)-5-propyl-octahydro-4,6-dioxo-pyrrolo[3,4-c]pyrrole-1-carboxylic acid;
rel-(1S,3R,3aS,6aR)-1-Isobutyl-2-(4-tert-butylbenzoyl)-3-(pyridin-2-yl)-5-propyl-octahydro-4,6-dioxo-pyrrolo[3,4-c]pyrrole-1-carboxylic acid;
rel-(1S,3R,3aS,6aR)-1-Isobutyl-2-(3-methoxy-4-tert-butylbenzoyl)-3-(1,3-thiazol-2-yl)-octahydro-4-oxo-5,5-dimethyl-cyclopenta[c]pyrrole-1-carboxylic acid;
rel-(1S,3R,3aS,6aR)-1-Isobutyl-2-(3-methoxy-4-tert-butylbenzoyl)-3-(5-methyl-1,3-thiazol-2-yl)-octahydro-4-oxo-cyclopenta[c]pyrrole-1-carboxylic acid;
and salts, solvates and enantiomers thereof.

Also included in the present invention are pharmaceutically acceptable salt complexes. The present invention also covers the physiologically acceptable salts of the compounds of formula (I). Suitable physiologically acceptable salts of the compounds of formula (I) include acid salts, for example sodium, potassium, calcium, magnesium and tetraalkylammonium and the like, or mono- or di-basic salts with the appropriate acid for example organic carboxylic acids such as acetic, lactic, tartaric, malic, isethionic, lactobionic and succinic acids; organic sulfonic acids such as methanesulfonic, ethanesulfonic, benzenesulfonic and p-toluenesulfonic acids and inorganic acids such as hydrochloric, sulfuric, phosphoric and sulfamic acids and the like.

The present invention also relates to solvates of the compounds of Formula (I), for example hydrates.

It will be appreciated that the compounds of the present invention may contain one or more asymmetric carbon atoms and may exist in racemic and optically active forms. All of these compounds and diastereomers are contemplated to be within the scope of the present invention.

It will further be appreciated that certain compounds of the present invention may exist in different tautomeric forms. All tautomers are contemplated to be within the scope of the present invention.

Compounds of Formula (I) may be prepared by reaction of a compound of Formula (II) 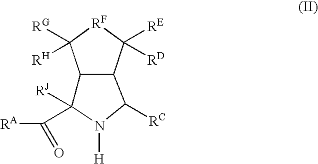
in which R^{A}, R^{C}, R^{D}, R^{E}, R^{F}, R^{G}, R^{H} and R^{J} are as defined above for Formula (I) with a suitable acylating agent; for example (R³CO)₂O or R³C(O)—X, wherein X is a halo atom, preferably chloro or bromo. Preferably the reaction is carried out in a suitable solvent, for example dichloromethane, tetrahydrofuran, acetonitrile or dimethylformamide in the presence of a suitable base, for example triethylamine, pyridine or diisopropylethylamine.

Compounds of Formula (II) may be prepared by reaction of a compound of Formula (III) 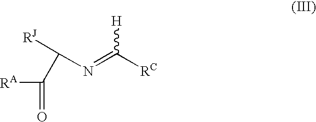
wherein R^{A}, R^{C}, and R^{J} are as defined for Formula (I) above; with a compound of Formula (IV) 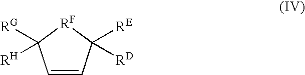
wherein R^{D}, R^{E}, R^{F}, R^{G} and R^{H} are as defined for Formula (I), providing that R^{D} and R^{E} together with the carbon atom to which they are attached, form a carbonyl group. Preferably, the reaction is carried out in a suitable solvent, for example tetrahydrofuran, dichloromethane, acetonitrile or dimethylformamide in the presence of a Lewis acid catalyst, such as lithium bromide or silver acetate and a base, such as triethylamine or diisopropylethylamine.

Alternatively, compounds of Formula (I) wherein R^{A} is OH may be prepared from a compound of Formula (V) 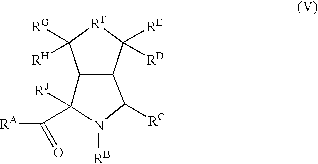
wherein R^{A} is OR¹ and R¹ is C₁₋₆alkyl, aryl, heteroaryl, arylalkyl or heteroarylalkyl and R^{B}, R^{C}, R^{D}, R^{E}, R^{F}, R^{G}, R^{H} and R^{J} are as defined above for Formula (I); for example when R¹ is tert-butyl, by treatment with an appropriate acid, for example trifluoroacetic acid. Suitably, the reaction is carried out in a solvent, for example dichloromethane. Preferably, the temperature is in the range 0 to 50° C., more preferably 15 to 30° C.

Compounds of Formula (V) may be prepared by reaction of a compound of Formula (IIa) 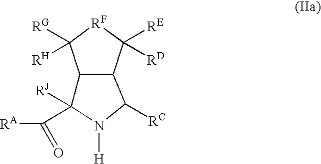
in which R^{A}, R^{C}, R^{D}, R^{E}, R^{F}, R^{G}, R^{H} and R^{J} are as defined above for Formula (V) with a suitable acylating agent; for example (R³CO)₂O or R³C(O)—X, wherein X is a halo atom, preferably chloro or bromo. Preferably the reaction is carried out in a suitable solvent, for example dichloromethane, tetrahydrofuran, acetonitrile or dimethylformamide in the presence of a suitable base, for example triethylamine, pyridine or diisopropylethylamine.

Compounds of Formula (IIa) may be prepared by reaction of a compound of Formula (IIIa) 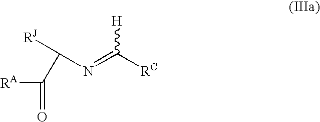
wherein R^{A}, R^{C}, and R^{J} are as defined for Formula (V) above; with a compound of Formula (IVa) 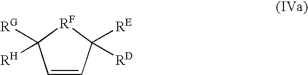
wherein R^{D}, R^{E}, R^{F}, R^{G} and R^{H} are as defined for Formula (V), providing that R^{D} and R^{E} together with the carbon atom to which they are attached, form a carbonyl group. Preferably, the reaction is carried out in a suitable solvent, for example tetrahydrofuran, dichloromethane, acetonitrile or dimethylformamide in the presence of a Lewis acid catalyst, such as lithium bromide or silver acetate and a base, such as triethylamine or diisopropylethylamine.

Compounds of Formula (I) in which R^{E} is OH may be prepared from compounds of Formula (I) in which R^{E} and R^{D} together with the carbon atom to which they are attached form a carbonyl group by treatment with a suitable reducing agent, such as lithium borohydride, diisobutylaluminium hydride, sodium borohydride with boron trifluoride etherate, in a suitable solvent such as tetrahydrofuran, diethylether or dichloromethane.

Compounds of Formula (II) in which R^{E} is OH may be prepared from compounds of Formula (II) in which R^{E} and R^{D} together with the carbon atom to which they are attached form a carbonyl group by treatment with a suitable reducing agent, such as lithium borohydride, diisobutylaluminium hydride, sodium borohydride with boron trifluoride etherate, in a suitable solvent such as tetrahydrofuran, diethylether or dichloromethane.

Compounds of Formula (I) in which R^{E} is OR⁴, and R⁴ is C₁₋₆alkyl or aryl, may be prepared from compounds of Formula (I) in which R^{E} is OH by treatment with a suitable acid, such as hydrochloric acid or p-toluenesulphonic acid, and an alcohol R⁴OH, in which R⁴ is C₁₋₆alkyl or aryl, optionally in a suitable solvent such as tetrahydrofuran, diethylether or dichloromethane.

Compounds of Formula (II) in which R^{E} is OR⁴, and R⁴ is C₁₋₆alkyl or aryl may be prepared from compounds of Formula (II) in which R^{E} is OH by treatment with a suitable acid, such as hydrochloric acid or p-toluenesulphonic acid, and an alcohol R⁴OH, in which R⁴ is C₁₋₆alkyl or aryl, optionally in a suitable solvent such as tetrahydrofuran, diethylether or dichloromethane.

Compounds of Formula (III) and (IV) are known in the art or may be prepared by standard literature procedures.

Compounds of Formula (II):
i) in which when R^{A} is OR¹ and OR¹ is OH, and
R^{C} is 4-fluorophenyl, 2,4-dichlorophenyl, 2-furanyl, 4-carboxymethylphenyl, 3-indolyl, 4-methoxyphenyl, 4-(dimethylamino)phenyl, 2-thienyl, 2-pyrrolyl or 2-pyridyl, and
R^{D} and R^{E} together with the carbon atom to which they are attached form a carbonyl group, and
R^{F} is 4-bromophenyl, phenyl or methyl, and
R^{G} and R^{H} together with the carbon atom to which they are attached form a carbonyl group, then
R^{J} is other than (1H-imidazol-5-yl)methyl, and
ii) which are other than
rel-(3aS,6aR)-1-((1H-imidazol-5-yl)methyl)-3-(2-furanyl)-octahydro-4,6-dioxo-pyrrolo[3,4-c]pyrrole-1-carboxylic acid; or
rel-(3aS,6aR)-1-((1H-imidazol-5-yl)methyl)-3-(2-pyrryl)-octahydro-4,6-dioxo-pyrrolo[3,4-c]pyrrole-1-carboxylic acid; or
rel-(1S,3R,3aS,6aR)-1-methyl-3-(4-methoxyphenyl)-5-phenyl-octahydro-4,6-dioxo-pyrrolo[3,4-c]pyrrole-1-carboxylic acid; or
rel-(1S,3R,3aS,6aR)-1-methyl-3-(4-nitrophenyl)-5-phenyl-octahydro-4,6-dioxo-pyrrolo[3,4-c]pyrrole-1-carboxylic acid; or
rel-(1S,3R,3aS,6aR)-1-benzyl-3-(4-methoxyphenyl)-5-phenyl-octahydro-4,6-dioxo-pyrrolo[3,4-c]pyrrole-1-carboxylic acid; or
rel-(1S,3R,3aS,6aR)-1-benzyl-3-(4-nitrophenyl)-5-phenyl-octahydro-4,6-dioxo-pyrrolo[3,4-c]pyrrole-1-carboxylic acid; or
rel-(1S,3R,3aS,6aR)-1-(2-methylpropyl)-3-(4-methoxyphenyl)-5-methyl-octahydro-4,6-dioxo-pyrrolo[3,4-c]pyrrole-1-carboxylic acid; or
rel-(1S,3R,3aS,6aR)-1-(2-methylpropyl)-3-(4-nitrophenyl)-5-methyl-octahydro-4,6-dioxo-pyrrolo[3,4-c]pyrrole-1-carboxylic acid; or
rel-(1S,3R,3aS,6aR)-1-methyl-3-(4-methoxyphenyl)-5-methyl-octahydro-4,6-dioxo-pyrrolo[3,4-c]pyrrole-1-carboxylic acid; or
rel-(1S,3R,3aS,6aR)-1-methyl-3-(4-nitrophenyl)-5-methyl-octahydro-4,6-dioxo-pyrrolo[3,4-c]pyrrole-1-carboxylic acid.
   are novel compounds useful as intermediates in the synthesis of compounds of Formula (I).

Compounds of Formula (V) in which R¹ is tert-butyl are novel compounds useful as intermediates in the synthesis of compounds of Formula (I).

With appropriate manipulation and protection of any chemical functionality, synthesis of compounds of Formula (I) is accomplished by methods analogous to those above and to those described in the Experimental section. Suitable protecting groups can be found, but are not restricted to, those found in T W Greene and P G M Wuts ‘Protective Groups in Organic Synthesis’, 3^{rd} Ed (1999), J Wiley and Sons.

### EXAMPLES

Intermediate 1

### 2-[N-(1,3-Thiazol-2-ylmethylene)amino]-4-methylpentanoic Acid, tert-butyl Ester

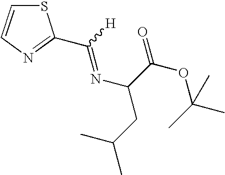

A stirred mixture of 2-amino-4-methyl-pentanoic acid tert-butyl ester, hydrochloride salt (5.00 g, 22.34 mmol), 1,3-thiazole-2-carboxaldehyde (2.53 g, 22.34 mmol) and triethylamine (3.10 mL, 22.3 mmol) in dichloromethane (60 mL) was heated under reflux under nitrogen for 19 hours. The reaction mixture was allowed to cool to room temperature, washed twice with water, dried over Na₂SO₄ and evaporated to give the title compound as an oil.

¹H NMR (CDCl₃): δ 8.46 (s, 1H), 7.94 (d, 1H), 7.44 (dd, 1H), 4.07 (dd, 1H), 1.89-1.74 (m, 2H), 1.64-1.52 (m, 1H), 1.48 (s, 9H), 0.96 (d, 3H) and 0.90 (d, 3H).

Intermediate 2

### rel-(1S,3R,3aS,6aR)-1-Isobutyl-3-(1,3-thiazol-2-yl)-5-propyl-octahydro-4,6-dioxo-pyrrolo[3,4-c]pyrrole-1-carboxylic Acid, Tert Butyl Ester

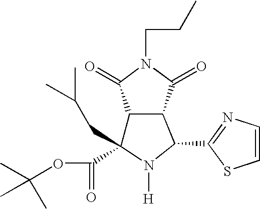

To a stirred solution of Intermediate 1 (2.05 g, 7.26 mmol) in anhydrous tetrahydrofuran (30 ml) under nitrogen, was added triethylamine (1.01 ml, 7.26 mmol) followed by lithium bromide (63 mg, 7.26 mmol) and a solution of N-propylmaleimide (1.01 g, 7.26 mmol) in anhydrous tetrahydrofuran (20 ml). The mixture was stirred at ambient temperature overnight. Aqueous ammonium chloride was added with rapid stirring and the resulting mixture was extracted twice with ethyl acetate. The combined organic layers were dried over sodium sulphate and evaporated to give the title compound as a solid.

Mass spec (electrospray) m/z calcd for (C₂₁H₃₁N₄O₄S+H)+: 422. Found: (M+H)+: 422

Intermediate 3

### rel-(1S,3R,3aS,6aR)-1-Isobutyl-3-(1,3-thiazol-2-yl)-5-benzyl-octahydro-4,6-dioxo-pyrrolo[3,4-c]pyrrole-1-carboxylic Acid, Tert Butyl Ester

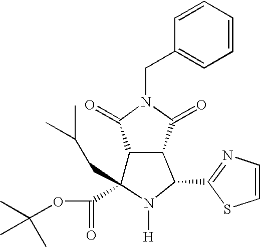

This was prepared following the procedure for Intermediate 2, replacing N-propylmaleimide with N-benzylmaleimide. The compound was purified by chromatography on silica gel using cyclohexane-ethyl acetate (4:1 v/v) as eluent to provide the title compound as a solid.

Mass spec (electrospray) m/z calc for (C₂₅H₃₁N₃O₄S+H)⁺: 470. Found: (M+H)⁺: 470

Intermediate 4

### rel-(1S,3R,3aS,6aR)-1-Isobutyl-3-(1,3-thiazol-2-yl)-5-methyl-octahydro-4,6-dioxo-pyrrolo[3,4-c]pyrrole-1-carboxylic Acid, Tert Butyl Ester

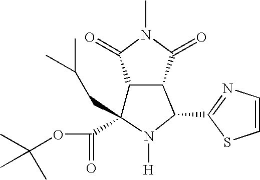

This was prepared following the procedure for Intermediate 2, replacing N-propylmaleimide with N-methylmaleimide to provide the title compound as a solid.

Mass spec (electrospray) m/z calc for (C₁₉H₂₇N₃O₄S+H)⁺: 394. Found: (M+H)⁺: 394

Intermediate 5

### rel-(1S,3R,3aS,6aR)-1-Isobutyl-3-(1,3-thiazol-2-yl)-octahydro-4-oxo-cyclopenta[c]pyrrole-1-carboxylic Acid, Tert Butyl Ester

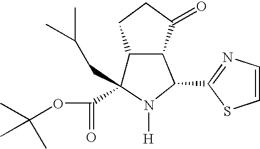

This was prepared following the procedure for Intermediate 2, replacing N-propylmaleimide with 2-cyclopentene-1-one, to provide the title compound as an oil.

Mass spec (electrospray) m/z calc for (C₁₉H₂₈N₂O₃S+H)⁺: 365. Found: (M+H)⁺: 365.

Intermediate 6

### rel-(3aR,4S,6R,6aS)-4-Isobutyl-6-(1,3-thiazol-2-yl)-hexahydro-furo[3,4-c]pyrrole-1-oxo-4-carboxylic Acid, Tert Butyl Ester

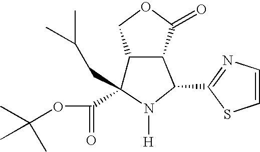

This was prepared following the procedure for Intermediate 2, replacing N-propylmaleimide with 2-(5H)-furanone, to provide the title compound as a solid.

Mass spec (electrospray) m/z calc for (C₁₈H₂₆N₂O₄S+H)⁺: 367. Found: (M+H)⁺: 367.

Intermediate 7

### rel-(1S,3R,3aS,6aR)-1-Isobutyl-2-(4-tert-butylbenzoyl)-3-(1,3-thiazol-2-yl)-5-propyl-octahydro-4,6-dioxo-pyrrolo[3,4-c]pyrrole-1-carboxylic Acid, Tert Butyl Ester

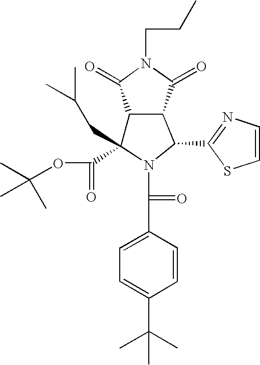

To a stirred solution of Intermediate 2 (1.5 g, 3.56 mmol) in anhydrous chloroform (60 mL) was added triethylamine (0.74 mL, 5.34 mmol) and 4-tert-butyl-benzoyl chloride (1 mL, 5.34 mmol). This mixture was stirred at ambient temperature for 18 hours. The mixture was then washed with water and extracted with dichloromethane. The organic phase was dried (Na₂SO₄) and evaporated.

The residue was purified by chromatography on silica gel using cyclohexane-ethyl acetate (1:1 v/v) as eluent to provide the title compound as a solid.

Mass spec (electrospray) m/z calcd for (C₃₂H₄₃N₃O₅S+H)⁺: 582. Found: (M+H)⁺=582.

Intermediate 8

### rel-(1S,3R,3aS,6aR)-1-Isobutyl-2-(4-tert-butylbenzoyl)-3-(1,3-thiazol-2-yl)-5-benzyl-octahydro-4,6-dioxo-pyrrolo[3,4-c]pyrrole-1-carboxylic Acid, Tert Butyl Ester

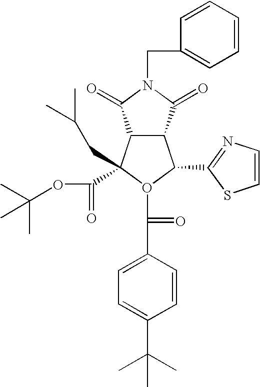

This was prepared following the procedure for Intermediate 7, replacing Intermediate 2 with Intermediate 3. The compound was purified by chromatography on silica gel using cyclohexane-ethyl acetate (4:1 v/v) as eluent to provide the title compound as a solid.

Mass spec (electrospray) m/z calc for (C₃₆H₄₃N₃O₅S+H)⁺: 630. Found: (M+H)⁺: 630

Intermediate 9

### rel-(1S,3R,3aS,6aR)-1-Isobutyl-2-(4-tert-butylbenzoyl)-3-(1,3-thiazol-2-yl)-5-methyl-octahydro-4,6-dioxo-pyrrolo[3,4-c]pyrrole-1-carboxylic Acid, Tert Butyl Ester

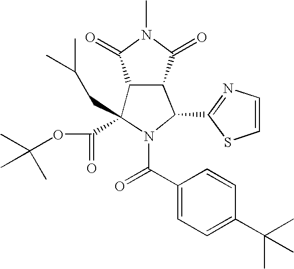

This was prepared following the procedure for Intermediate 7 replacing Intermediate 2 with Intermediate 4. The compound was purified by chromatography on silica gel using cyclohexane-ethyl acetate (1:1 v/v) as eluent to provide the title compound as a solid.

Mass spec (electrospray) m/z calc for (C₃₀H₃₉N₃O₅S+H)⁺: 554. Found: (M+H)⁺: 554.

Intermediate 10

### rel-(1S,3R,3aS,6aR)-1-Isobutyl-2-(4-tert-butylbenzoyl)-3-(1,3-thiazol-2-yl)-octahydro-4-oxo-cyclopenta[c]pyrrole-1-carboxylic Acid, Tert Butyl Ester

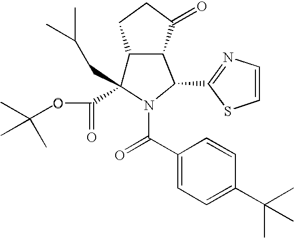

This was prepared following the procedure for Intermediate 7, replacing Intermediate 2 with Intermediate 5. The compound was purified by chromatography on silica gel using cyclohexane-ethyl acetate (4:1 v/v) as eluent to provide the title compound as a solid.

Mass spec (electrospray) m/z calc for (C₃₀H₄₀N₂O₄S+H)⁺: 525. Found: (M+H)⁺: 525.

Intermediate 11

### rel-(3aR,4S,6R,6aS)-4-Isobutyl-5-(4-tert-butylbenzoyl)-6-(1,3-thiazol-2-yl)-hexahydro-furo[3,4-c]pyrrol-1-oxo-4-carboxylic Acid, Tert Butyl Ester

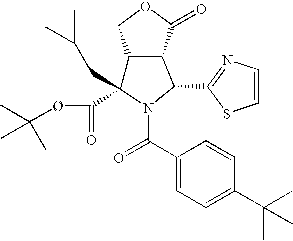

This was prepared following the procedure for Intermediate 7, replacing Intermediate 2 with Intermediate 6. The compound was purified by chromatography on silica gel using cyclohexane-ethyl acetate (3:2 v/v) as eluent to provide the title compound as a solid.

Mass spec (electrospray) m/z calc for (C₂₉H₃₈N₂O₅S+H)⁺: 527. Found: (M+H)⁺: 527.

Intermediate 12

### rel-(1S,3R,3aS,6aR)-1-Isobutyl-2-(3-methoxy-4-tert-butylbenzoyl)-3-(1,3-thiazol-2-yl)-octahydro-4-oxo-cyclopenta[c]pyrrole-1-carboxylic Acid, Tert Butyl Ester

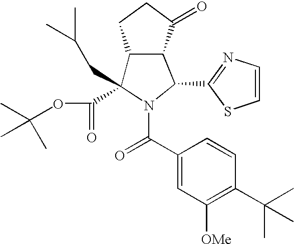

To a solution of Intermediate 5 (0.750 g, 2.06 mmol) in dichloromethane (15 mL) was added 3-methoxy-4-tert-butylbenzoyl chloride* (0.560 mg, 2.47 mmol) followed by triethylamine (0.36 mL, 2.58 mmol) and the mixture stirred at room temperature for 18 hours. The solvents were removed in vacuo and the residue purified by chromatography using a SPE cartridge (10 g silica) and eluting with cyclohexane, then cyclohexane-ethyl acetate (3:2, v/v). This afforded the title compound as a foam.
*{Synthesized from 4-tert-butyl-3-methoxybenzoic acid (J. Org. Chem, 26, 1961,1732-1737)}.

MS calcd for (C₃₁H₄₂N₂O₅S+H)⁺: 555

MS found (electrospray): (M+H)⁺=555

Intermediate 13

### rel-(1S,3R,3aS,6aR)-1-Isobutyl-2-(3-bromo-4-tert-butylbenzoyl)-3-(1,3-thiazol-2-yl)-5-propyl-octahydro-4,6-dioxo-pyrrolo[3,4-c]pyrrole-1-carboxylic Acid, Tert Butyl Ester

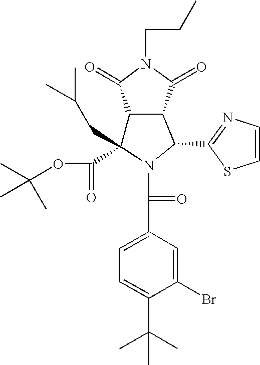

To a stirred solution of Intermediate 2 (0.3 g, 0.71 mmol) in dry dichloromethane (2 mL) was added 3-bromo-4-tert-butylbenzoyl chloride* (0.215 g, 1.1 eq.) dissolved in dry dichloromethane (3 mL), followed by triethylamine (0.123 mL, 0.89 mmol). This mixture was stirred at ambient temperature for 18 hours. The mixture was then diluted with dichloromethane (8 mL) and washed with water. The organic phase was dried (Na₂SO₄) and evaporated.
*Synthesised from 3-bromo-4-tert-butylbenzoic acid (Aust. J. Chem. (1990), 43(5), 807-14).

The residue was purified by chromatography on silica gel using cyclohexane-ethyl acetate (4:1 v/v) as eluent to provide the title compound as a solid.

¹H NMR (CDCl₃): δ 7.36 (d, 1H), 7.25 (d,1H), 7.17 (d,1H), 7.14 (d,1H), 7.04 (dd, 1H), 6.02 (d, 1H), 4.00 (t, 1H), 3.58 (d, 1H), 3.28 (m, 2H), 2.74 (dd, 1H), 2.13 (dd, 1H), 1.79 (m, 1H), 1.60 (s, 9H), 1.50 (m, 2H), 1.43 (s, 9H), 1.16 (d, 3H), 1.14 (d, 3H), 0.87 (t, 3H).

Intermediate 14

### 2-[N-(Pyridin-2-ylmethylene)amino]-4-methylpentanoic Acid, Tert-Butyl Ester

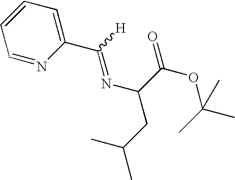

A stirred mixture of 2-amino-4-methylpentanoic acid tert-butyl ester hydrochloride (5.00 g, 22.35 mmol), pyridine-2-carboxaldehyde (2.12 mL, 22.35 mmol) and triethylamine (3.11 mL, 22.35 mmol) in dichloromethane (75 mL) was heated under reflux under nitrogen for 2 hours. The reaction mixture was allowed to cool to room temperature, washed with water and brine, dried over MgSO₄ and evaporated to give the title compound as an oil.

¹H NMR (CDCl₃): δ 8.65 (ddd, 1H), 8.37 (s, 1H), 8.12 (dt, 1H), 7.75 (ddt, 1H), 7.34 (ddd, 1H), 4.05 (dd, 1H), 1.79-1.85 (m, 2H), 1.58 (m, 1H), 1.47 (s, 9H), 0.95 (d, 3H) and 0.91 (d, 3H).

Intermediate 15

### rel-(1S,3R,3aS,6aR)-1-Isobutyl-3-(pyridin-2-yl)-5-propyl-octahydro-4,6-dioxo-pyrrolo[3,4-c]pyrrole-1-carboxylic Acid, Tert Butyl Ester

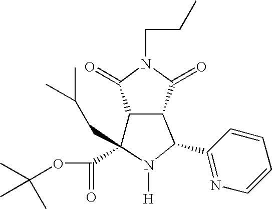

This was prepared following the procedure for Intermediate 2, replacing Intermediate 1 with Intermediate 14. This afforded the title compound as a solid.

Mass spec (electrospray) m/z calc for (C₂₃H₃₃N₃O₄+H)⁺: 416. Found (M+H)⁺: 416.

Intermediate 16

### rel-(1S,3R,3aS,6aR)-1-Isobutyl-2-(3-bromo-4-tert-butylbenzoyl)-3-(pyridin-2-yl)-5-propyl-octahydro-4,6-dioxo-pyrrolo[3,4-c]pyrrole-1-carboxylic Acid, Tert Butyl Ester

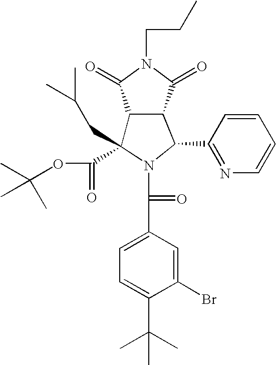

To a stirred solution of Intermediate 15 (0.269 g, 0.65 mmol) in dry dichloromethane (3 mL) was added 3-bromo-4-tert-butylbenzoyl chloride* (0.215 g, 1.1 eq.) dissolved in dry dichloromethane (1 mL), followed by triethylamine (0.113 mL, 0.81 mmol). This mixture was stirred at ambient temperature for 2 days. The mixture was then diluted with dichloromethane (8 mL) and washed with water (5 mL). The organic phase was dried (Na₂SO₄) and evaporated.

The residue was purified by chromatography on silica gel using cyclohexane-ethyl acetate (4:1, v/v) as eluent to provide the title compound as a solid.

¹H NMR (CDCl₃): δ 81.4(d, 1H), 7.70 (d, 1H), 7.45 (dt, 1H), 7.16 (d, 1H), 6.95-7.04 (m, 3H), 5.67 (d, 1H), 4.00 (dd, 1H), 3.55 (d, 1H), 3.23 (m, 2H), 2.78 (dd, 1H), d.13 (dd, 1H), 1.81 (m, 1H), 1.60 (s, 9H), 1.45-1.55 (m, 2H), 1.37 (s, 9H), 1.16 (d, 3H), 1.15 (d, 3H), 0.83 (t, 3H).

Intermediate 17

### rel-(1S,3R,3aS,6aR)-1-Isobutyl-2-(4-tert-butylbenzoyl)-3-(pyridin-2-yl)-5-propyl-octahydro-4,6-dioxo-pyrrolo[3,4-c]pyrrole-1-carboxylic Acid, Tert Butyl Ester

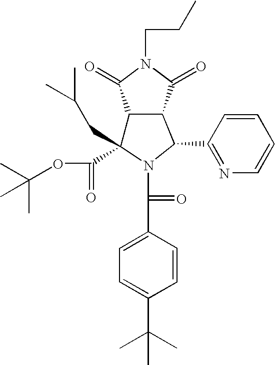

This was prepared following the procedure for Intermediate 16, replacing 3-bromo-4-tert-butylbenzoyl chloride with 4-tert-butylbenzoyl chloride. This afforded the title compound as a solid.

¹H NMR (CDCl₃): δ 8.09 (d, 1H), 7.71 (d, 1H), 7.38 (dt, 1H), 7.05 (d, 2H), 6.97 (d, 2H), 6.88 (m, 1H), 5.77 (d, 1H), 3.99 (dd, 1H), 3.56 (d, 1H), 3.22 (m, 2H), 2.81 (dd, 1H), 2.12 (dd, 1H), 1.84 (m, 1H), 1.60 (s, 9H), 1.31-1.46 (m, 2H), 1.17 (s, 9H), 1.16 (d, 3H), 1.14 (d, 3H), 0.82 (t, 3H).

Intermediate 18

### rel-(1S,3R,3aS,6aR)-1-Isobutyl-3-(1,3-thiazol-2-yl)-octahydro-4-oxo-5,5dimethyl-cyclopenta[c]pyrrole-1-carboxylic Acid, Tert Butyl Ester

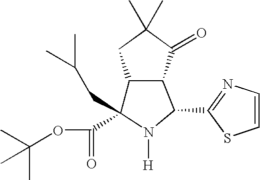

This was prepared following the procedure for Intermediate 2, replacing N-propylmaleimide with 5,5-dimethyl-2-cyclopentene-1-one*, to provide the title compound as an oil.
*Prepared by the method described in Tetrahedron (1993) 49, 7931.

Mass spec (electrospray) m/z calc for (C₂₁H₃₂N₂O₃S+H)⁺: 393. Found: (M+H)⁺: 393.

Intermediate 19

### rel-(1S,3R,3aS,6aR)-1-Isobutyl-2-(4-tert-butylbenzoyl)-3-(1,3-thiazol-2-yl)-octahydro-4-oxo-5,5-dimethyl-cyclopenta[c]pyrrole-1-carboxylic Acid, Tert Butyl Ester

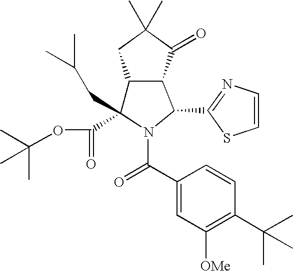

This was prepared following the procedure for Intermediate 12, replacing Intermediate 7 with Intermediate 18. The compound was purified by chromatography on silica gel using cyclohexane-ethyl acetate (10:1 then 4:1, v/v) as eluent to provide the title compound as a solid.

Mass spec (electrospray) m/z calc for (C₃₃H₄₆N₂O₅S+H)⁺: 583. Found: (M+H)⁺: 583.

Intermediate 20

### 2-[N-(5-methyl-thiazol-2-ylmethylene)amino]-4-methylpentanoic Acid, Tert-Butyl Ester

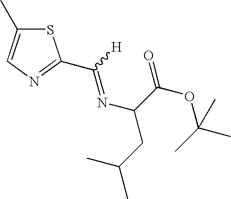

This was prepared following the procedure for Intermediate 1, replacing thiazole-2-carboxaldehyde with 5-methyl-4-hiazole-2-carboxaldehyde to provide the title compound.

¹H NMR (CDCl₃): δ 8.33 (s, 1H), 7.56 (s, 1H), 4.01 (m, 1H), 2.49 (s, 3H), 1.75 (m, 2H), 1.52 (m, 1H), 1.45 (s, 9H), 0.93 (d, 3H) and 0.88 (d, 3H).

Intermediate 21

### rel-(1S,3R,3aS,6aR)-1-Isobutyl-3-(5-methyl-1,3-thiazol-2-yl)-octahydro-4-oxo cyclopenta[c]pyrrole-1-carboxylic Acid, Tert Butyl Ester

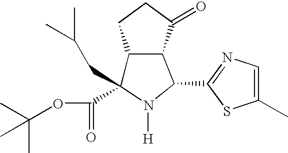

This was prepared following the procedure for Intermediate 2, replacing N-propylmaleimide with cyclopentenone and Intermediate 1 with Intermediate 20 to provide the title compound.

Mass spec (electrospray) m/z calc for (C₂₀H₃₀N₂O₃S+H)⁺: 379. Found: (M+H)⁺: 379.

Intermediate 22

### rel-1 S,3R,3aS,6aR)-1-Isobutyl-2-(3-methoxy-4-tert-butylbenzoyl)-3-(5-methyl-1,3-thiazol-2-yl)-octahydro-4-oxo-cyclopenta[c]pyrrole-1-carboxylic Acid, Tert Butyl Ester

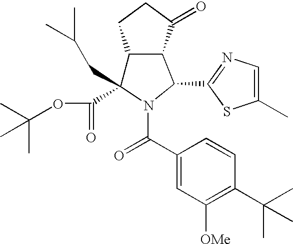

This was prepared following the procedure for Intermediate 12, replacing Intermediate 5 with Intermediate 21 to provide the title compound.

Mass spec (electrospray) m/z calc for (C₃₂H₄₄N₂O₅S+H)⁺: 569. Found: (M+H)⁺: 569.

### Example 1

### rel-(1 S,3R,3aS,6aR)-1-Isobutyl-2-(4-tert-butylbenzoyl)-3-(1,3-thiazol-2-yl)-5-propyl-octahydro-4,6-dioxo-pyrrolo[3,4-c]pyrrole-1-carboxylic Acid

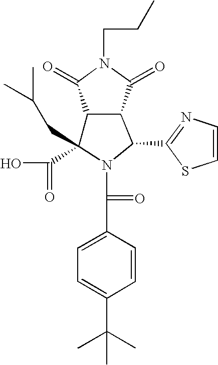

To a solution of Intermediate 7 (44 mg, 8.85 mmol) in dichloromethane (5 mL) was added trifluoroacetic acid (1 mL) and the solution stirred at ambient temperature overnight. The reaction mixture was evaporated and the residue triturated with diethylether to give the title compound as a solid.

Mass spec (electrospray) m/z calc for (C₂₈H₃₅N₂O₅S+H)⁺: 526. Found: (M+H)⁺ 526

### Example 2

### rel-(1S,3R,3aS,6aR)-1-Isobutyl-2-(4-tert-butylbenzoyl)-3-(1,3-thiazol-2-yl)-5-benzyl-octahydro-4,6-dioxo-pyrrolo[3,4-c]pyrrole-1-carboxylic Acid

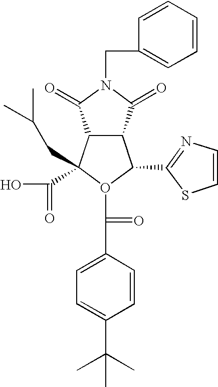

This was prepared following the procedure for Example 1, replacing Intermediate 7 with Intermediate 8. The title compound was prepared as a solid.

Mass spec (electrospray) m/z calc for (C₃₂H₃₅N₃O₅S+H)⁺: 574. Found: (M+H)⁺: 574

### Example 3

### rel-(1S,3R,3aS,6aR)-1-Isobutyl-2-(4-tert-butylbenzoyl)-3-(1,3-thiazol-2-yl)-5-methyl-octahydro-4,6-dioxo-pyrrolo[3,4-c]pyrrole-1-carboxylic Acid

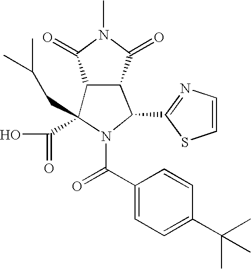

This was prepared following the procedure for Example 1, replacing Intermediate 7 with Intermediate 9. The title compound was prepared as a solid.

Mass spec (electrospray) m/z calc for (C₂₆H₃₁N₃O₅S+H)⁺: 498. Found: (M+H)⁺: 498.

### Example 4

### rel-(1S,3R,3aS,6aR)-1-Isobutyl-2-(4-tert-butylbenzoyl)-3-(1,3-thiazol-2-yl)-octahydro-4-oxo-cyclopenta[c]pyrrole-1-carboxylic Acid

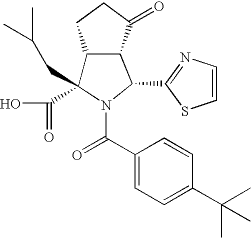

This was prepared following the procedure for Example 1, replacing Intermediate 7 with Intermediate 10. The title compound was prepared as a solid.

Mass spec (electrospray) m/z calc for (C₂₆H₃₂N₂O₄S+H)⁺: 469. Found: (M+H)⁺: 469.

### Example 5

### rel-(3aR,4S,6R,6aS)-4-Isobutyl-5-(4-tert-butylbenzoyl)-6-(1,3-thiazol-2-yl)-hexahydro-furo[3,4-c]pyrrol-1-oxo-4-carboxylic Acid

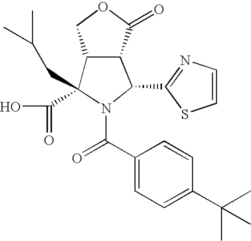

This was prepared following the procedure for Example 1 replacing Intermediate 7 with Intermediate 11. The title compound was prepared as a solid.

Mass spec (electrospray) m/z calc for (C₂₅H₃₀N₂O₅S+H)⁺: 471. Found: (M+H)⁺: 471.

### Example 6

### rel-(1S,3R,3aS,6aR)-1-Isobutyl-2-(3-methoxy-4-tert-butylbenzoyl)-3-(1,3-thiazol-2-yl)-octahydro-4-oxo-cyclopenta[c]pyrrole-1-carboxylic Acid

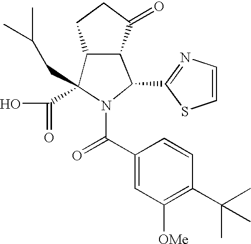

This was prepared following the procedure for Example 1, replacing Intermediate 7 with Intermediate 12.

MS calcd for (C₂₇H₃₄N₂O₅S+H)⁺: 499.

MS found (electrospray): (M+H)⁺=499.

### Example 7

### rel-(1S,3R,3aS,6aR)-1-Isobutyl-2-(3-bromo-4-tert-butylbenzoyl)-3-(1,3-thiazol-2-yl)-5-propyl-octahydro-4,6-dioxo-pyrrolo[3,4-c]pyrrole-1-carboxylic Acid

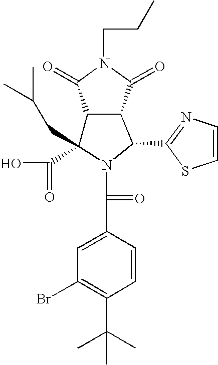

This was prepared following the procedure for Example 1, replacing Intermediate 7 with Intermediate 13.

MS calcd for (C₂₈H₃₄BrN₃O₅S+H)⁺: 604, 606

MS found (electrospray): (M+H)⁺=604, 606

### Example 8

### Enantiomer A of rel-(1S,3R,3aS,6aR)-1-Isobutyl-2-(3-bromo-4-tert-butylbenzoyl)-3-(1,3-thiazol-2-yl)-5-propyl-octahydro-4,6-dioxo-pyrrolo[3,4-c]pyrrole-1-carboxylic Acid

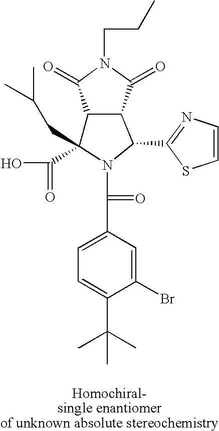 Step A

Intermediate 13 was resolved by preparative HPLC on a Chiralcel OD-H chromatography column using heptane-ethanol (80:20, v/v) as eluent to afford the individual enantiomers with retention times of 5.9 minutes (Enantiomer A of rel-(1S,3R,3aS,6aR)-1-Isobutyl-2-(3-bromo-4-tert-butylbenzoyl)-3-(1,3-thiazol-2-yl)-5-propyl-octahydro-4,6-dioxo-pyrrolo[3,4-c]pyrrole-1-carboxylic acid, tert butyl ester) and 4.0 minutes (Enantiomer B) respectively. Enantiomer A was identical by ¹H NMR to Intermediate 13.

Step B

Enantiomer A of rel-(1S,3R,3aS,6aR)-1-Isobutyl-2-(3-bromo-4-tert-butylbenzoyl)-3-(1,3-thiazol-2-yl)-5-propyl-octahydro-4,6-dioxo-pyrrolo[3,4-c]pyrrole-1-carboxylic acid, tert butyl ester was deprotected following the procedure described for Example 1, replacing Intermediate 7 with Enantiomer A of Intermediate 13 to provide the title compound as a solid.

MS calcd for (C₂₈H₃₄BrN₃O₅S+H)⁺: 604, 606.

MS found (electrospray): (M+H)⁺=604, 606.

### Example 9

### Enantiomer A of rel-(1S,3R,3aS,6aR)-1-Isobutyl-2-(3-bromo-4-tert-butylbenzoyl)-3-(1,3-pyridin-2-yl)-5-propyl-octahydro-4,6-dioxo-pyrrolo[3,4-c]pyrrole-1-carboxylic Acid

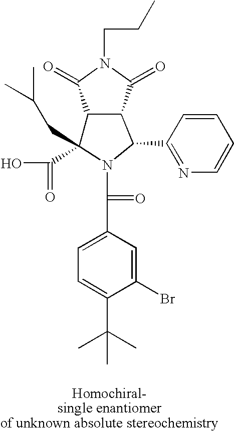 Step A

Intermediate 16 was resolved by preparative HPLC on a Chiralpak AD chromatography column using heptane-isopropanol (60:40, v/v) as eluent to afford the individual enantiomers with retention times of 4.9 minutes (Enantiomer A of rel-(1S,3R,3aS,6aR)-1-Isobutyl-2-(3-bromo-4-tert-butylbenzoyl)-3-(pyridin-2-yl)-5-propyl-octahydro-4,6-dioxo-pyrrolo[3,4-c]pyrrole-1-carboxylic acid, tert butyl ester) and 9.4 minutes (Enantiomer B) respectively. Enantiomer A was identical by ¹H NMR to intermediate 16.

Step B

Enantiomer A of rel-(1S,3R,3aS,6aR)-1-Isobutyl-2-(3-bromo-4-tert-butylbenzoyl)-3-(pyridin-2-yl)-5-propyl-octahydro-4,6-dioxo-pyrrolo[3,4-c]pyrrole-1-carboxylic acid, tert butyl ester was deprotected following the procedure described for Example 1, replacing Intermediate 7 with Enantiomer A of Intermediate 16 to provide the title compound as a solid.

Mass spec (electrospray) m/z calc for (C₃₀H₃₆BrN₃O₅+H)⁺: 598, 600. Found: (M+H)⁺: 598, 600.

### Example 10

### Enantiomer A of rel-(1S,3R,3aS,6aR)-1-Isobutyl-2-(4-tert-butylbenzoyl)-3-(pyridin-2-yl)-5-propyl-octahydro-4,6-dioxo-pyrrolo[3,4-c]pyrrole-1-carboxylic Acid

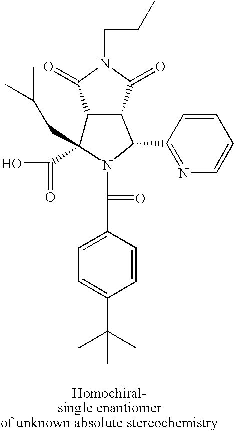 Step A

Intermediate 17 was resolved by preparative HPLC on a Chiralpak AD chromatography column using heptane-isopropanol (70:30, v/v) as eluent to afford the individual enantiomers with retention times of 10.24 minutes (Enantiomer A of rel-(1S,3R,3aS,6aR)-1-Isobutyl-2-(4-tert-butylbenzoyl)-3-(pyridin-2-yl)-5-propyl-octahydro-4,6-dioxo-pyrrolo[3,4-c]pyrrole-1-carboxylic acid, tert butyl ester) and 6.82 minutes (Enantiomer B) respectively. Enantiomer A was identical by ¹H NMR to Intermediate 17.

Step B

Enantiomer A of rel-(1S,3R,3aS,6aR)-1-Isobutyl-2-(4-tert-butylbenzoyl)-3-(pyridin-2-yl)-5-propyl-octahydro-4,6-dioxo-pyrrolo[3,4-c]pyrrole-1-carboxylic acid, tert butyl ester was deprotected following the procedure described for Example 1, replacing Intermediate 7 with Enantiomer A of Intermediate 17 to provide the title compound as a solid.

Mass spec (electrospray) m/z calc for (C₃₀H₃₇N₃O₅+H)⁺: 520. Found: (M+H)⁺: 520.

### Example 11

### Enantiomer A of rel-(1S,3R,3aS,6aR)-1-Isobutyl-2-(4-tert-butylbenzoyl)-3-(1,3-thiazol-2-yl)-5-propyl-octahydro-4,6-dioxo-pyrrolo[3,4-c]pyrrole-1-carboxylic Acid

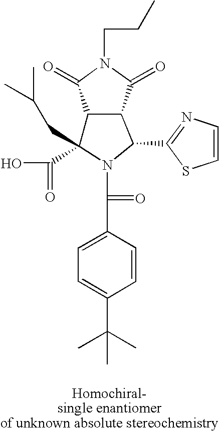 Step A

Intermediate 7 was resolved by preparative HPLC on a Chiralcel OD chromatography column using heptane-isopropanol (85:15, v/v) as eluent to afford the individual enantiomers with retention times of 11 minutes (Enantiomer A of rel-(1S,3R,3aS,6aR)-1-Isobutyl-2-(4-tert-butylbenzoyl)-3-(thiazol-2-yl)-5-propyl-octahydro-4,6-dioxo-pyrrolo[3,4-c]pyrrole-1-carboxylic acid, tert butyl ester) and 4.5 minutes (Enantiomer B) respectively. Enantiomer A was identical by ¹H NMR to Intermediate 7.

Step B

Enantiomer A of rel-(1S,3R,3aS,6aR)-1-Isobutyl-2-(4-tert-butylbenzoyl)-3-(1,3-thiazol-2-yl)-5-propyl-octahydro-4,6-dioxo-pyrrolo[3,4-c]pyrrole-1-carboxylic acid, tert butyl ester was deprotected following the procedure described for Example 1, replacing

Intermediate 7 with Enantiomer A of Intermediate 7 to provide the title compound as a solid.

¹H NMR (CDCl₃): δ 7.65 (d, 1H), 7.2 (m, 3H), 7.0 (d, 2H), 5.95 (d, 1H), 4.1 (t, 1H), 3.7 (d, 1H), 3.2 (t, 2H), 2.65 (dd, 1H), 2.4 (dd, 1H), 1.6 (m, 1H), 1.60 (s, 9H), 1.25 (s, 9H), 1.2 (m, 1H), 1.15 (2xd, 6H), 1.1 (m, 1H), 0.75 (t, 3H).

### Example 12

### rel-(1S,3R,3aS,6aR)-1-Isobutyl-2-(3-methoxy-4-ert-butylbenzoyl)-3-(1,3-thiazol-2-yl)-octahydro-4-oxo-5,5-dimethyl-cyclopenta[c]pyrrole-1-carboxylic Acid

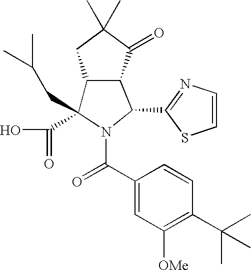

This was prepared following the procedure for Example 1, replacing Intermediate 7 with Intermediate 19.

MS calcd for (C₂₉H₃₈N₂O₆S+H)⁺: 527.

MS found (electrospray): (M+H)⁺=527.

### Example 13

### rel-(1S,3R,3aS,6aR)-1-Isobutyl-2-(3-methoxy-4-tert-butylbenzoyl)-3-(5-methyl-1,3-thiazol-2-yl)-octahydro-4-oxo-cyclopenta[c]pyrrole-1-carboxylic Acid

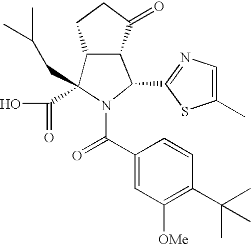

This was prepared following the procedure for Example 1, replacing Intermediate 7 with Intermediate 22.

MS calcd for (C₂₈H₃₆N₂O₅S+H)⁺: 513.

MS found (electrospray): (M+H)⁺=513.

The compounds according to the invention may be formulated for administration in any convenient way, and the invention therefore also includes within its scope pharmaceutical compositions for use in therapy, comprising a compound of formula (I) or a physiologically acceptable salt, solvate or enantiomer thereof in admixture with one or more physiologically acceptable diluents or carriers.

The compounds of the present invention can be administered by different routes including intravenous, intraperitoneal, subcutaneous, intramuscular, oral, topical, transdermal, or transmucosal administration. For systemic administration, oral administration is preferred. For oral administration, for example, the compounds can be formulated into conventional oral dosage forms such as capsules, tablets and liquid preparations such as syrups, elixirs and concentrated drops.

Alternatively, injection (parenteral administration) may be used, e.g., intramuscular, intravenous, intraperitoneal, and subcutaneous. For injection, the compounds of the invention are formulated in liquid solutions, preferably, in physiologically compatible buffers or solutions, such as saline solution, Hank's solution, or Ringer's solution. In addition, the compounds may be formulated in solid form and redissolved or suspended immediately prior to use. Lyophilized forms can also be produced.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, bile salts and fusidic acid derivatives. In addition, detergents may be used to facilitate permeation. Transmucosal administration, for example, may be through nasal sprays, rectal suppositories, or vaginal suppositories.

For topical administration, the compounds of the invention can be formulated into ointments, salves, gels, or creams, as is generally known in the art.

The amounts of various compounds to be administered can be determined by standard procedures taking into account factors such as the compound (IC₅₀) potency, (EC₅₀) efficacy, and the biological half-life (of the compound), the age, size and weight of the patient, and the disease or disorder associated with the patient. The importance of these and other factors to be considered are known to those of ordinary skill in the art.

Amounts administered also depend on the routes of administration and the degree of oral bioavailability. For example, for compounds with low oral bioavailability, relatively higher doses will have to be administered. Oral administration is a preferred method of administration of the present compounds.

Preferably the composition is in unit dosage form. For oral application, for example, a tablet, or capsule may be administered, for nasal application, a metered aerosol dose may be administered, for transdermal application, a topical formulation or patch may be administered and for transmucosal delivery, a buccal patch may be administered. In each case, dosing is such that the patient may administer a single dose.

Each dosage unit for oral administration contains suitably from 0.01 to 500 mg/Kg, and preferably from 0.1 to 50 mg/Kg, of a compound of Formula (I) or a pharmaceutically acceptable salt thereof, calculated as the free base. The daily dosage for parenteral, nasal, oral inhalation, transmucosal or transdermal routes contains suitably from 0.01 mg to 100 mg/Kg, of a compound of Formula (I). A topical formulation contains suitably 0.01 to 5.0% of a compound of Formula (I). The active ingredient may be administered from 1 to 6 times per day, preferably once, sufficient to exhibit the desired activity, as is readily apparent to one skilled in the art.

Composition of Formula (I) and their pharmaceutically acceptable salts which are active when given orally can be formulated as syrups, tablets, capsules and lozenges. A syrup formulation will generally consist of a suspension or solution of the compound or salt in a liquid carrier for example, ethanol, peanut oil. olive oil, glycerine or water with a flavoring or coloring agent. Where the composition is in the form of a tablet, any pharmaceutical carrier routinely used for preparing solid formulations may be used. Examples of such carriers include magnesium stearate, terra alba, talc, gelatin, acacia, stearic acid, starch, lactose and sucrose. Where the composition is in the form of a capsule, any routine encapsulation is suitable, for example using the aforementioned carriers in a hard gelatin capsule shell. Where the composition is in the form of a soft gelatin shell capsule any pharmaceutical carrier routinely used for preparing dispersions or suspensions may be considered, for example aqueous gums, celluloses, silicates or oils, and are incorporated in a soft gelatin capsule shell.

Typical parenteral compositions consist of a solution or suspension of a compound or salt in a sterile aqueous or non-aqueous carrier optionally containing a parenterally acceptable oil, for example polyethylene glycol, polyvinylpyrrolidone, lecithin, arachis oil or sesame oil.

Typical compositions for inhalation are in the form of a solution, suspension or emulsion that may be administered as a dry powder or in the form of an aerosol using a conventional propellant such as dichlorodifluoromethane or trichlorofluoromethane.

A typical suppository formulation comprises a compound of Formula (I) or a pharmaceutically acceptable salt thereof which is active when administered in this way, with a binding and/or lubricating agent, for example polymeric glycols, gelatins, cocoa-butter or other low melting vegetable waxes or fats or their synthetic analogs.

Typical dermal and transdermal formulations comprise a conventional aqueous or non-aqueous vehicle, for example a cream, ointment, lotion or paste or are in the form of a medicated plaster, patch or membrane.

No unacceptable toxological effects are expected when compounds of the present invention are administered in accordance with the present invention.

### Assay

The potential for compounds of the invention to inhibit NS5B wildtype HCV polymerase activity may be demonstrated, for example, using the following in vitro assay:

In Vitro Detection of Inhibitors of HCV RNA-Dependent RNA Polymerase Activity

Incorporation of [3H]-UMP into RNA was followed by absorption of the RNA polymer onto a DEAE glass fibre filter. A synthetic template consisting of 16mer oligoU hybridised to polyrA (10:1 w/w) was used as a homopolymer substrate.

Reaction Conditions were 22 μM [³H]-UTP (0.75 Ci/mmol), 1 mM-Dithiothreitol, 3.2 mM-MgCl₂, 20 mM-Tris-HCl, pH7.0, 10 μg/mL polyA-oligoU, and 90 mM-NaCl. Note that 50 mM-NaCl is added with the enzyme.

HCV RNA Polymerase (Recombinant full-length NS5B (Lohmann et al., J. Virol. 71 (11), 1997, 8416 ‘Biochemical properties of hepatitis C virus NS5B RNA-dependent RNA polymerase and identification of amino acid sequence motifs essential for enzymatic activity’) expressed in baculovirus and purified to homogeneity) was diluted to about 50 μg protein/mL (dependent on specific activity) in 50 mM-Hepes, pH7.0, 0.5M-NaCl, 20%-Glycerol, 0.05%-Triton X-100, 5 mM-Dithiothreitol, 0.1 mM-EDTA.

5× Concentrated Buffer mix was prepared using 1M-Tris-HCl (pH7.0, 1 mL), 1M-MgCl₂ (0.16 mL), 1M-Dithiothreitol (0.05 mL), 5M-NaCl (0.4 mL), and Water (8.4 mL), Total 10 mL.

Substrate Mix was prepared using 5× Concentrated Buffer mix (12 μL), [3H]-UTP (1 μCi/μL; 21.7 μM, 1 μL), 22 μM-UTP (100 μM, 13.2 μL), 10 μg/mL polyA-oligoU (100 μg/mL, 6 μL), and Water (12.8 μL), Total 45 μL.

The Assay was set up using Substrate Mix (45 μL), compound (10 μL), and Diluted Enzyme (added last to start reaction) (5 μL), Total 60 μL.

The reaction was performed in a U-bottomed, clear, 96-well plate. The reaction was mixed on a plate-shaker, after addition of the Enzyme, and incubated for 2 h at 22° C. After this time, the reaction was stopped by addition of 25 μL of 100 mM-EDTA.

A DEAE Filtermat (Part No. 1205405 from Pharmacia) was pre-washed in water and alcohol and dried. 2×20 μL of the Stopped Assay Mix was spotted onto a square of the DEAE Filtermat. The DEAE Filtermat was washed for 2×15 min in SSC buffer (0.3M-NaCl, 30 mM-Na Citrate) followed by 2×2 min in water and 1×1 min in alcohol. The Filtermat was dried and sealed in a bag together with 10 mL of OptiScint HiSafe scintillation fluid. The radioactivity present on the filtermat was detected by scintillation counting on a Wallac 1205 Betaplate counter. After subtraction of background levels without enzyme, any reduction in the amount of radioactivity incorporated in the presence of a compound, compared to that in the absence, was taken as a measure of the level of inhibition. Ten concentrations of compounds were tested in two- or threefold dilutions. From the counts, percentage of inhibition at highest concentration tested or IC₅₀s for the compounds were calculated using Grafit3 or Grafit4 software packages.

The exemplified compounds had an IC₅₀ of <50 μM. Accordingly, the compounds of the invention are of potential therapeutic benefit in the treatment and prophylaxis of HCV.

All publications, including but not limited to patents and patent applications cited in this specification are herein incorporated by reference as if each individual publication were specifically and individually indicated to be incorporated by reference as though fully set forth.

1. A compound of Formula (I) 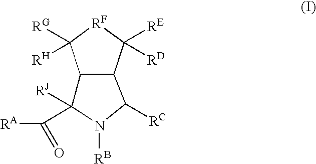
wherein:
R^{A} represents OR¹, NR¹R², or R¹ wherein R¹ and R² independently represent hydrogen, C₁₋₆alkyl, aryl, heteroaryl, arylalkyl or heteroarylalkyl; or R¹ and R² together with the nitrogen atom to which they are attached form a 5 or 6 membered saturated cyclic group;
R^{B} represents C(O)R³ wherein R³ represents aryl or heteroaryl;
R^{C} represents C₁₋₆alkyl, aryl, heteroaryl or heterocyclyl;
R^{D} represents hydrogen and R^{E} represents hydrogen, OR⁴ or SR⁴, or R^{D} and R^{E} together with the carbon atom to which they are attached form a carbonyl group or a thiocarbonyl group;
when R^{E} is hydrogen, OR⁴ or SR⁴, R^{G} and R^{H} are both hydrogen;
when R^{D} and R^{E} together with the carbon atom to which they are attached form a carbonyl group or a thiocarbonyl group, R^{G} represents hydrogen and R^{H} represents hydrogen, OR⁴ or SR⁴, or R^{G} and R^{H} together with the carbon atom to which they are attached form a carbonyl group or a thiocarbonyl group;
R⁴ represents hydrogen, C₁₋₆alkyl or aryl;
when R^{D} and R^{E} together with the carbon atom to which they are attached form a carbonyl group or a thiocarbonyl group, and R^{G} and R^{H} are both hydrogen or R^{G} and R^{H} together with the carbon atom to which they are attached form a carbonyl group or a thiocarbonyl group, then R^{F} represents O, S, NR⁵ or CR⁶R⁷, otherwise R^{F} represents CR⁶R⁷;
R⁵ represents hydrogen, C₁₋₆alkyl, arylalkyl or aryl;
R⁶ and R⁷ independently represent hydrogen, C₁₋₆alkyl, arylalkyl or heteroarylalkyl;
R^{J} represents hydrogen, C₁₋₆alkyl, heterocyclylalkyl, arylalkyl or heteroarylalkyl;
and salts, solvates and enantiomers thereof;
provided that when R^{A} is OR¹ then R¹ is other than tert-butyl.

2. Compounds of Formula (I), represented by Formula (A) 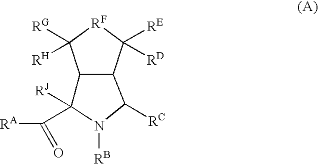
wherein:
R^{A} represents OR¹, NR¹R², or R¹ wherein R¹ and R² independently represent hydrogen, C₁₋₆alkyl, aryl, heteroaryl, arylalkyl or heteroarylalkyl; or R¹ and R² together with the nitrogen atom to which they are attached form a 5 or 6 membered saturated cyclic group;
R^{B} represents C(O)R³ wherein R³ represents aryl or heteroaryl;
R^{C} represents C₁₋₆alkyl, aryl, heteroaryl or heterocyclyl;
R^{D} represents hydrogen and R^{E} represents hydrogen, OR⁴ or SR⁴, or R^{D} and R^{E} together with the carbon atom to which they are attached form a carbonyl group or a thiocarbonyl group;
when R^{E} is hydrogen, OR⁴ or SR⁴, R^{G} and R^{H} are both hydrogen;
when R^{D} and R^{E} together with the carbon atom to which they are attached form a carbonyl group or a thiocarbonyl group, R^{G} represents hydrogen and R^{H} represents hydrogen, OR⁴ or SR⁴, or R^{G} and R^{H} together with the carbon atom to which they are attached form a carbonyl group or a thiocarbonyl group;
R⁴ represents hydrogen, C₁₋₆alkyl or aryl;
when R^{D} and R^{E} together with the carbon atom to which they are attached form a carbonyl group or a thiocarbonyl group, and R^{G} and R^{H} are both hydrogen or R^{G} and R^{H} together with the carbon atom to which they are attached form a carbonyl group or a thiocarbonyl group, then R^{F} represents O, S, NR¹ or CR⁶R⁷, otherwise R^{F} represents CR⁶R⁷;
R⁵ represents hydrogen, C₁₋₆alkyl, arylalkyl or aryl;
R⁶ and R⁷ independently represent hydrogen, C₁₋₆alkyl, arylalkyl or heteroarylalkyl;
R^{J} represents hydrogen, C₁₋₆alkyl, heterocyclylalkyl, arylalkyl or heteroarylalkyl;
and salts, solvates and enantiomers thereof; provided that
i) when R^{A} is OR¹ then R¹ is other than tert-butyl, and P1 ii) a compound of Formula (A) is other than
rel-(3aS,6aR)-1-((1H-imidazol-5-yl)methyl)-2-(cyclopropylbenzoyl)-3-(4-methoxyphenyl)-5-methyl-octahydro-4,6-dioxo-pyrrolo[3,4-c]pyrrole-1-carboxylic acid; or
rel-(1S,3R,3aS,6aR)-1-methyl-2-benzoyl-3-(4-methoxyphenyl)-5-phenyl-octahydro-4,6-dioxo-pyrrolo[3,4-c]pyrrole-1-carboxylic acid; or
rel-(1S,3R,3aS,6aR)-1-methyl-2-benzoyl-3-(4-nitrophenyl)-5-methyl-octahydro-4,6-dioxo-pyrrolo[3,4-c]pyrrole-1-carboxylic acid; or
rel-(1S,3R,3aS,6aR)-1-benzyl-2-(phenylethanoyl)-3-(4-methoxyphenyl)-5-methyl-octahydro-4,6-dioxo-pyrrolo[3,4-c]pyrrole-1-carboxylic acid; or
rel-(1S,3R,3aS,6aR)-1-(2-methylpropyl)-2-ethanoyl-3-(4-methoxyphenyl)-5-methyl-octahydro-4,6-dioxo-pyrrolo[3,4-c]pyrrole-1-carboxylic acid; or
rel-(1S,3R,3aS,6aR)-1-methyl-2-ethanoyl-3-(4-methoxyphenyl)-5-methyl-octahydro-4,6-dioxo-pyrrolo[3,4-c]pyrrole-1-carboxylic acid.

3. A compound as claimed in claim 1 wherein R^{A} is OR¹.

4. A compound as claimed in claim 1 wherein R^{A} is OH.

5. A compound as claimed claim 1 wherein R³ is phenyl, optionally substituted by halo, C₁₋₆alkyl or C₁₋₃alkoxy.

6. A compound as claimed claim 1 wherein R³ is 4-tert-butylphenyl optionally 3-substituted by halo, C₁₋₃alkyl or C₁₋₃alkoxy.

7. A compound as claimed claim 1 wherein R³ is 4-tert-butylphenyl, 3-bromo-4-tert-butylphenyl or 4-tert-butyl-3-methoxyphenyl.

8. A compound as claimed claim 1 wherein R^{C} is heteroaryl.

9. A compound as claimed claim 1 wherein R^{C} is 1,3-thiaol-2-yl or pyridin-2-yl.

10. A compound as claimed in claim 1 selected from the group consisting of:
rel-(1S,3R,3aS,6aR)-1-Isobutyl-2-(4-tert-butylbenzoyl)-3-(1,3-thiazol-2-yl)-5-propyl-octahydro-4,6-dioxo-pyrrolo[3,4-c]pyrrole-1-carboxylic acid;
rel-(1S,3R,3aS,6aR)-1-Isobutyl-2-(4-tert-butylbenzoyl)-3-(1,3-thiazol-2-yl)-5-benzyl-octahydro-4,6-dioxo-pyrrolo[3,4-c]pyrrole-1-carboxylic acid;
rel-(1S,3R,3aS,6aR)-1-Isobutyl-2-(4-tert-butylbenzoyl)-3-(1,3-thiazol-2-yl)-5-methyl-octahydro-4,6-dioxo-pyrrolo[3,4-c]pyrrole-1-carboxylic acid;
rel-(1S,3R,3aS,6aR)-1-Isobutyl-2-(4-tert-butylbenzoyl)-3-(1,3-thiazol-2-yl)-octahydro-4-oxo-cyclopenta[c]pyrrole-1-carboxylic acid;
rel-(3aR,4S,6R,6aS)-4-Isobutyl-5-(4-tert-butylbenzoyl)-6-(1,3-thiazol-2-yl)-hexahydro-furo[3,4-c]pyrrol-1-oxo-4-carboxylic acid;
rel-(1S,3R,3aS,6aR)-1-Isobutyl-2-(3-methoxy-4-tert-butylbenzoyl)-3-(1,3-thiazol-2-yl)-octahydro-4-oxo-cyclopenta[c]pyrrole-1-carboxylic acid;
rel-(1S,3R,3aS,6aR)-1-Isobutyl-2-(3-bromo-4-tert-butylbenzoyl)-3-(1,3-thiazol-2-yl)-5-propyl-octahydro-4,6-dioxo-pyrrolo[3,4-c]pyrrole-1-carboxylic acid;
rel-(1S,3R,3aS,6aR)-1-Isobutyl-2-(3-bromo-4-tert-butylbenzoyl)-3-(1,3-pyridin-2-yl)-5-propyl-octahydro-4,6-dioxo-pyrrolo[3,4-c]pyrrole-1-carboxylic acid;
rel-(1S,3R,3aS,6aR)-1-Isobutyl-2-(4-tert-butylbenzoyl)-3-(pyridin-2-yl)-5-propyl-octahydro-4,6-dioxo-pyrrolo[3,4-c]pyrrole-1-carboxylic acid;
rel-(1S,3R,3aS,6aR)-1-Isobutyl-2-(3-methoxy-4-tert-butylbenzoyl)-3-(1,3-thiazol-2-yl)-octahydro-4-oxo-5,5-dimethyl-cyclopenta[c]pyrrole-1-carboxylic acid;
rel-(1 S,3R,3aS,6aR)-1-Isobutyl-2-(3-methoxy-4-tert-butylbenzoyl)-3-(5-methyl-1,3-thiazol-2-yl)-octahydro-4-oxo-cyclopenta[c]pyrrole-1-carboxylic acid;
and salts, solvates and enantiomers thereof.

11. A method for the treatment of a human or animal subject with viral infection, comprising administering to said human or animal subject an effective amount of a compound of Formula (I) 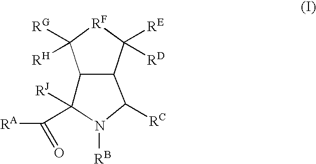
wherein:
R^{A} represents OR¹, NR¹R², or R¹ wherein R¹ and R² independently represent hydrogen, C₁₋₆alkyl, aryl, heteroaryl, arylalkyl or heteroarylalkyl; or R¹ and R² together with the nitrogen atom to which they are attached form a 5 or 6 membered saturated cyclic group;
R^{B} represents C(O)R³ wherein R³ represents aryl or heteroaryl;
R^{C} represents C₁₋₆alkyl, aryl, heteroaryl or heterocyclyl;
R^{D} represents hydrogen and R^{E} represents hydrogen, OR⁴ or SR⁴, or R^{D} and R^{E} together with the carbon atom to which they are attached form a carbonyl group or a thiocarbonyl group;
when R^{E} is hydrogen, OR⁴ or SR⁴, R^{G} and R^{H} are both hydrogen;
when R^{D} and R^{E} together with the carbon atom to which they are attached form a carbonyl group or a thiocarbonyl group, R^{G} represents hydrogen and R^{H} represents hydrogen, OR⁴ or SR⁴, or R^{G} and R^{H} together with the carbon atom to which they are attached form a carbonyl group or a thiocarbonyl group;
R⁴ represents hydrogen, C₁₋₆alkyl or aryl;
when R^{D} and R^{E} together with the carbon atom to which they are attached form a carbonyl group or a thiocarbonyl group, and R^{G} and R^{H} are both hydrogen or R^{G} and R^{H} together with the carbon atom to which they are attached form a carbonyl group or a thiocarbonyl group, then R^{F} represents O, S, NR⁵ or CR⁶R⁷, otherwise R^{F} represents CR⁶R⁷;
R⁵ represents hydrogen, C₁₋₆alkyl, arylalkyl or aryl;
R⁶ and R⁷ independently represent hydrogen, C₁₋₆alkyl, arylalkyl or heteroarylalkyl;
R^{J} represents hydrogen, C₁₋₆alkyl, heterocyclylalkyl, arylalkyl or heteroarylalkyl;
and salts, solvates and enantiomers thereof;
provided that when R^{A} is OR¹ then R¹ is other than tert-butyl.

12. A method as claimed in claim 11 wherein the viral infection is HCV.

13. (canceled)

14. (canceled)

15. (canceled)

16. (canceled)

17. A pharmaceutical composition comprising a compound as claimed in claim 1 or a physiologically acceptable salt, solvate or enantiomer thereof in admixture with one or more physiologically acceptable diluents or carriers.

18. A process for the preparation of compounds of Formula (I) as claimed in claim 1, comprising reaction of a compound of Formula (II) 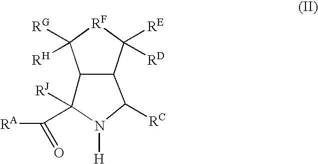
in which R^{A}, R^{C}, R^{D}, R^{E}, R^{F}, R^{G}, R^{H} and R^{J} are as defined for Formula (I) with an acylating agent.

19. A process for the preparation of compounds of Formula (I) wherein R^{A} is OH, comprising hydrolysis of a compound of Formula (V) 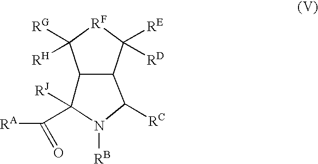
wherein R^{A} is OR¹ and R¹ is C₁₋₆alkyl, aryl, heteroaryl, arylalkyl or heteroarylalkyl and R^{B}, R^{C}, R^{D}, R^{E}, R^{F}, R^{G}, R^{H} and R^{J} are as defined in claim 1 for Formula (I), with an acid.

20. Intermediate compounds of Formula (II) 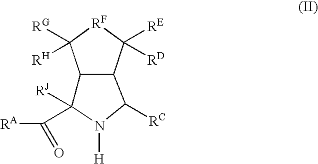
wherein
R^{A} represents OR¹, NR¹R², or R¹ wherein R¹ and R² independently represent hydrogen, C₁₋₆alkyl, aryl, heteroaryl, arylalkyl or heteroarylalkyl; or R¹ and R² together with the nitrogen atom to which they are attached form a 5 or 6 membered saturated cyclic group;
R^{C} represents C₁₋₆alkyl, aryl, heteroaryl or heterocyclyl;
R^{D} represents hydrogen and R^{E} represents hydrogen, OR⁴ or SR⁴, or R^{D} and R^{E} together with the carbon atom to which they are attached form a carbonyl group or a thiocarbonyl group;
when R^{E} is hydrogen, OR⁴ or SR⁴, R^{G} and R^{H} are both hydrogen;
when R^{D} and R^{E} together with the carbon atom to which they are attached form a carbonyl group or a thiocarbonyl group, R^{G} represents hydrogen and R^{H} represents hydrogen, OR⁴ or SR⁴, or R^{G} and R^{H} together with the carbon atom to which they are attached form a carbonyl group or a thiocarbonyl group;
R⁴ represents hydrogen, C₁₋₆alkyl or aryl;
when R^{D} and R^{E} together with the carbon atom to which they are attached form a carbonyl group or a thiocarbonyl group, and R^{G} and R^{H} are both hydrogen or R^{G} and R^{H} together with the carbon atom to which they are attached form a carbonyl group or a thiocarbonyl group, then R^{F} represents O, S, NR⁵ or CR⁶R⁷, otherwise R^{F} represents CR⁶R⁷;
R⁵ represents hydrogen, C₁₋₆alkyl, arylalkyl or aryl;
R⁶ and R⁷ independently represent hydrogen, C₁₋₆alkyl, arylalkyl or heteroarylalkyl;
R^{J} represents hydrogen, C₁₋₆alkyl, heterocyclylalkyl, arylalkyl or heteroarylalkyl; provided that
i) when R^{A} is OR¹ and OR¹ is OH, and
R^{C} is 4-fluorophenyl, 2,4-dichlorophenyl, 2-furanyl, 4-carboxymethylphenyl, 3-indolyl, 4-methoxyphenyl, 4-(dimethylamino)phenyl, 2-thienyl, 2-pyrrolyl or 2-pyridyl, and
R^{D} and R^{E} together with the carbon atom to which they are attached form a carbonyl group, and
R^{F} is 4-bromophenyl, phenyl or methyl, and
R^{G} and R^{H} together with the carbon atom to which they are attached form a carbonyl group, then
R^{J} is other than (1H-imidazol-5-yl)methyl, and
ii) a compound of Formula (II) is other than
rel-(3aS,6aR)-1-((1H-imidazol-5-yl)methyl)-3-(2-furanyl)-octahydro-4,6-dioxo-pyrrolo[3,4-c]pyrrole-1-carboxylic acid; or
rel-(3aS,6aR)-1-((1H-imidazol-5-yl)methyl)-3-(2-pyrryl)-octahydro-4,6-dioxo-pyrrolo[3,4-c]pyrrole-1-carboxylic acid; or
rel-(1S,3R,3aS,6aR)-1-methyl-3-(4-methoxyphenyl)-5-phenyl-octahydro-4,6-dioxo-pyrrolo[3,4-c]pyrrole-1-carboxylic acid; or
rel-(1S,3R,3aS,6aR)-1-methyl-3-(4-nitrophenyl)-5-phenyl-octahydro-4,6-dioxo-pyrrolo[3,4-c]pyrrole-1-carboxylic acid; or
rel-(1S,3R,3aS,6aR)-1-benzyl-3-(4-methoxyphenyl)-5-phenyl-octahydro-4,6-dioxo-pyrrolo[3,4-c]pyrrole-1-carboxylic acid; or
rel-(1S,3R,3aS,6aR)-1-benzyl-3-(4-nitrophenyl)-5-phenyl-octahydro-4,6-dioxo-pyrrolo[3,4-c]pyrrole-1-carboxylic acid; or
rel-(1S,3R,3aS,6aR)-1-(2-methylpropyl)-3-(4-methoxyphenyl)-5-methyl-octahydro-4,6-dioxo-pyrrolo[3,4-c]pyrrole-1-carboxylic acid; or
rel-(1S,3R,3aS,6aR)-1-(2-methylpropyl)-3-(4-nitrophenyl)-5-methyl-octahydro-4,6-dioxo-pyrrolo[3,4-c]pyrrole-1-carboxylic acid; or
rel-(1S,3R,3aS,6aR)-1-methyl-3-(4-methoxyphenyl)-5-methyl-octahydro-4,6-dioxo-pyrrolo[3,4-c]pyrrole-1-carboxylic acid; or
rel-(1S,3R,3aS,6aR)-1-methyl-3-(4-nitrophenyl)-5-methyl-octahydro-4,6-dioxo-pyrrolo[3,4-c]pyrrole-1-carboxylic acid.

21. Intermediate compounds of Formula (V) 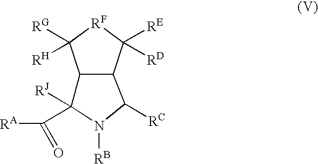
wherein R^{A} is OR¹ and R¹ is C₁₋₆alkyl, aryl, heteroaryl, arylalkyl or heteroarylalkyl and R^{B}, R^{C}, R^{D}, R^{E}, R^{F}, R^{G}, R^{H} and R^{J} are as defined above for Formula (I).
